(19) United States
(12) Patent Application Publication
(10) Pub. No.: US 2024/0327853 A1
(43) Pub. Date: Oct. 3, 2024

(54) DOUBLE DECAPITATION OF PLANTS

(71) Applicant: Keygene N.V., Wageningen (NL)

(72) Inventors: Bjorn Alexander KLOOSTERMAN, Wageningen (NL); Franck Georges Paul LHUISSIER, Wageningen (NL)

(21) Appl. No.: 18/743,454

(22) Filed: Jun. 14, 2024

Related U.S. Application Data
(63) Continuation of application No. PCT/EP2022/086257, filed on Dec. 16, 2022.

(30) Foreign Application Priority Data
Dec. 17, 2021 (EP) 21215549.3

(51) Int. Cl. C12N 15/82; C12N 9/22; C12N 15/11

(52) U.S. Cl. CPC C12N 15/8213; C12N 9/22; C12N 15/111; C12N 15/8203; C12N 2310/20; C12N 2800/80

(57) ABSTRACT

The invention pertains to a method for producing a shoot of a plant, wherein the shoot comprises one or more cells having a genetic modification. The method preferably comprises the steps of: i) providing a plant, ii) introducing in a cell of the plant a vector expressing at least one of a gRNA and a CRISPR-nuclease, iii) removing the shoot apical meristem of the plant, and iv) regenerating a shoot at a second location in the plant, wherein the shoot comprises one or more cells having a genetic modification. The shoot apical meristem of the plant is preferably removed by decapitating the plant. Similarly, the shoot is preferably regenerated by a second decapitation of the plant at a second location. The invention further related to a plant having a shoot comprising one or more cells having a genetic modification, wherein the plant is obtainable by the method of the invention.

### CROSS-REFERENCE TO RELATED APPLICATION

This application is a continuation of International PCT Application PCT/EP2022/086257 filed Dec. 16, 2022, which application claims priority to European Patent Application No. 21215549.3, filed Dec. 17, 2021, the entire contents of which are hereby incorporated by reference herein.

### SEQUENCE LISTING

The instant application contains a Sequence Listing which has been submitted in ASCII format via EFS-WEB and is hereby incorporated by reference in its entirety. Said ASCII copy, created on Jun. 13, 2024, is named 085342-4800_Sequence_listing.txt and is 32 KB.

### FIELD OF THE INVENTION

The present invention relates to the field of molecular plant biology and plant production. The invention concerns the targeted genetic modification of a plant cell. In particular, the invention concerns a shoot comprising one or more cells having a targeted genetic modification, as well as plants and plant products derived therefrom.

### BACKGROUND

Plant breeding aims to improve the productivity and performance of plants through selection and recombination of useful and superior traits, and to improve plant traits by genetic technologies. These technologies are faced with a central technical challenge: how to regenerate from a modified single cell back to a fertile plant. This is true both for techniques aimed at making genetic changes (genome editing, mutagenesis, genetic transformation) and for those affecting the genome as a whole (DH production, polyploidisation, somatic hybridisations).

An essential step in plant regeneration is the formation of one or more new shoots. Prior to regeneration, the plant may be exposed to a vector that introduces a heritable modification in one or more plant cells. These modified cells may subsequently form part of the newly regenerated shoot. This technique is however fairly inefficient and only a limited number of the newly regenerated shoots comprise the heritable mutation. Also, the number of modified cells that are present in those shoots is quite low. There is therefore still a strong need in the art to improve the capability of a plant to form shoots comprising one or more cells having a heritable modification.

### SUMMARY

The method may be summarized in the following embodiments:

Embodiment 1. A method for producing a shoot of a plant, wherein the shoot comprises one or more cells having a genetic modification, and wherein the method comprises the steps of:
i) providing a plant;
ii) introducing in a cell of the plant a vector expressing at least one of a guide element and nuclease element of a site-directed nuclease;
iii) removing the apical meristem of the plant, preferably by decapitating the plant at a first location; and
iv) regenerating a shoot at a second location in the plant, preferably by wounding the plant at the second location, wherein the shoot comprises one or more cells having a genetic modification.

Embodiment 2. A method according to embodiment 1, wherein the shoot regeneration is by decapitation of the plant at the second location.

Embodiment 3. The method according to embodiment 1 or 2, wherein the first location in step iii) is above the cotyledons, preferably in the epicotyl above the cotyledons and below the first true leaves.

Embodiment 4. The method according to any one of the preceding embodiments, wherein the second location in step iv) is below the cotyledons, preferably in the hypocotyl below the cotyledons.

Embodiment 5. The method according to any one of the preceding embodiments, wherein the site-directed nuclease is selected from the group consisting of a CRISPR-nuclease complex, an Argonaute, a Zinc finger nuclease, a TALEN and a meganuclease.

Embodiment 6. The method according to any one of the preceding embodiments, wherein the site-specific nuclease is a CRISPR-nuclease complex, and wherein the plant provided in step i) expresses the nuclease element of said complex and wherein in step ii) the vector expresses a gRNA.

Embodiment 7. The method according to any one of the preceding embodiments, wherein the period between step ii) and step iii) is about 1-21 days.

Embodiment 8. The method according to any one of the preceding embodiments, wherein the period between step iii) and step iv) is about 1-7 days.

Embodiment 9. The method according to any one of the preceding embodiments, wherein the plant provided in step i) is a seedling.

Embodiment 10. The method according to any one of the preceding embodiments, wherein the introduced vector in step ii) is a virus, preferably selected from the group consisting of a Tobacco Rattle Virus (TRV), a Tobacco Mosaic Virus (TMV), a Potato virus X (PVX), a geminivirus and a Sonchus yellow net virus (SYNV), preferably a tobacco mosaic virus RNA-based overexpression vector (TRBO).

Embodiment 11. The method according to any one of the preceding embodiments, wherein the vector is introduced in step ii) by contacting the cotyledon with an agrobacterium or virus particle carrying said vector.

Embodiment 12. The method according to any one of the preceding embodiments, further comprising a step of selecting the shoot comprising one or more cells having a genetic modification and regenerating a plant thereof.

Embodiment 13. The method according to any one of the preceding embodiments, wherein the method further comprises a step of growing the plant to produce a gamete comprising the genetic modification.

Embodiment 14. The method according to embodiment 13, wherein the method further comprises a step of growing a seed from said gamete and optionally a progeny plant from said seed, wherein the seed and optional progeny plant comprises one or more cells having the genetic modification.

Embodiment 15. A plant having a shoot comprising one or more cells having a genetic modification, wherein the plant is obtainable by the method according to any one of embodiments 1-14.

### Definitions

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. It is clear for the skilled person that any methods and materials similar or equivalent to those described herein can be used for practising the present invention.

Methods of carrying out the conventional techniques used in methods of the invention will be evident to the skilled worker. The practice of conventional techniques in molecular biology, biochemistry, computational chemistry, cell culture, recombinant DNA, bioinformatics, genomics, sequencing and related fields are well-known to those of skill in the art and are discussed, for example, in the following literature references: Sambrook et al. Molecular Cloning. A Laboratory Manual, 2nd Edition, Cold Spring Harbor Laboratory Press, Cold Spring Harbor, N. Y., 1989; Ausubel et al. Current Protocols in Molecular Biology, John Wiley & Sons, New York, 1987 and periodic updates; and the series Methods in Enzymology, Academic Press, San Diego.

The singular terms “a” “an” and “the” include plural referents unless the context clearly dictates otherwise. Thus, for example, reference to “a cell” includes a combination of two or more cells, and the like. The indefinite article “a” or “an” thus usually means “at least one”.

As used herein, the term “about” is used to describe and account for small variations. For example, the term can refer to less than or equal to ± (+ or −) 10%, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

“Analogous to” in respect of a domain, sequence or position of a protein, in relation to an indicated domain, sequence or position of a reference protein, is to be understood herein as a domain, sequence or position that aligns to the indicated domain, sequence or position of the reference protein, upon alignment of the protein to the reference protein using alignment algorithms as described herein, such as Needleman Wunsch.

The term “and/or” refers to a situation wherein one or more of the stated cases may occur, alone or in combination with at least one of the stated cases, up to with all of the stated cases.

The term “cDNA” means complementary DNA. Complementary DNA is made by reverse transcribing RNA into a complementary DNA sequence. cDNA sequences thus correspond to RNA sequences that are expressed from genes. As RNA sequences expressed from the genome can undergo splicing, i.e. introns are spliced out of the pre-mRNA and exons are joined together, before being translated in the cytoplasm into proteins, it is understood that the sequence of the cDNA corresponds to the sequence of the mRNA. The cDNA sequence thus may not be identical to the genomic DNA sequence to which it corresponds as the cDNA may encode only the complete open reading frame, consisting of the joined exons, for a protein, whereas the genomic DNA sequence may comprise exon sequences interspersed by intron sequences. Genetically modifying a gene which encodes a protein may thus not only relate to modifying the sequences encoding the protein, but may also involve mutating intronic sequences of the genomic DNA and/or other gene regulatory sequences of that gene.

The term “comprising” is construed as being inclusive and open ended, and not exclusive. Specifically, the term and variations thereof mean the specified features, steps or components are included. These terms are not to be interpreted to exclude the presence of other features, steps or components.

The term “conditions that allow for regeneration” is herein understood as an environment wherein a plant cell or tissue can regenerate. Such conditions include at minimum a suitable temperature (i.e. between 0° C.-60° C.), nutrition, day/night rhythm, irrigation and one or more plant hormones and/or plant hormone-like compounds. Furthermore, “optimal conditions that allow for regeneration” are those environmental conditions that allow for a maximum regeneration of the plant cells.

A “control plant” as referred to herein is a plant of the same species and preferably same genetic background as the plant of the invention, i.e. a plant that has been subjected to the methods as taught herein. Preferably the control plant is grown under the same conditions as the plant subjected to a method of the invention.

A “CRISPR-nuclease” or “CAS-protein” comprises two nuclease domains and is capable of complexing with a guide RNA. When complexed with a guide RNA, the CRISPR-nuclease complex is directed to a specific nucleic acid sequence by a guide RNA. In case both domains of the nuclease are catalytically active the protein is able to introduce a double-stranded break at the target site. The guide RNA interacts with said CRISPR-nuclease as well as with a target-specific nucleic acid sequence, indicated herein as the protospacer sequence, such that, once directed to the site comprising the target nucleic acid sequence via the guide sequence, the CRISPR-nuclease is able to introduce a double-stranded break at or near the target site. In case the CRISPR-nuclease has one domain that is catalytically active and one domain is catalytically inactive the CRISPR-nuclease is indicated herein as a CRISPR-nickase. Said CRISPR-nickase is able to introduce a single-stranded break at the target site. In case the CRISPR-nuclease is a dead CRISPR-nuclease, both domains are catalytically inactive, and the protein is unable to introduce a break at the target site. The skilled person is well aware of how to design a guide RNA in a manner that it, when combined with a CRISPR-nuclease having endonuclease or nickase activity, effects the introduction of a double- or single-stranded break, respectively, at a predefined site in the nucleic acid molecule. CRISPR-nucleases can generally be categorized into six major types (Type I-VI), which are further subdivided into subtypes, based on core element content and sequences (Makarova et al, 2011, Nat Rev Microbiol 9:467-77 and Wright et al, 2016, Cell 164 (1-2):29-44). In general, the two key elements of a CRISPR-nuclease complex are a CRISPR-nuclease and a guide RNA. Type II CRISPR-nuclease complexes include a signature Cas9 protein, a single protein (about 160 KDa), capable of binding a guide RNA and specifically cleaving duplex DNA. The Cas9 protein typically contains two active nuclease domains, a RuvC-like nuclease domain near the amino terminus and the HNH (or McrA-like) nuclease domain near the middle of the protein. Each nuclease domain of the Cas9 protein is specialized for cutting one strand of the double helix (Jinek et al, 2012, Science 337 (6096):816-821). The Cas9 protein is an example of a CRISPR-nuclease of the type II CRISPR-CAS system and forms an site-specific endonuclease, when combined with the crRNA and a second RNA termed the trans-activating crRNA (tracrRNA). The crRNA and tracrRNA function together as the guide RNA. The CRISPR-nuclease complex introduces DNA double strand breaks (DSBs) at the position in the genome defined by the crRNA. Jinek et al. (2012, Science 337:816-820) demonstrated that a single chain chimeric guide RNA (herein defined as a “sgRNA” or “single guide RNA”) produced by fusing an essential portion of the crRNA and tracrRNA was able to form a functional CRISPR-nuclease complex when combined with the Cas9 protein. A Type V CRISPR-nuclease complex has been described, the Clustered Regularly Interspaced Short Palindromic Repeats from Prevotella and Francisella, or CRISPR/Cpf1. Cpf1 genes are associated with the CRISPR locus and code for an endonuclease that use a crRNA to target DNA. Cpf1 is a smaller endonuclease than Cas9, which may overcome some of the CRISPR-Cas9 limitations. Cpf1 is a single RNA-guided endonuclease lacking a tracrRNA, and it utilizes a T-rich protospacer-adjacent motif. Cpf1 cleaves DNA via a staggered DNA double-stranded break (Zetsche et al (2015) Cell 163 (3):759-771). The type V CRISPR-nuclease system preferably includes at least one of Cpf1, C2c1 and C2c3. The part of the crRNA that is complementary to the protospacer sequence is designed to have sufficient complementarity with the protospacer sequence to hybridize with the protospacer sequence and direct sequence-specific binding of a complexed CRISPR protein. The protospacer sequence is preferably adjacent to a protospacer adjacent motif (PAM) sequence, which PAM sequence may interact with the CRISPR protein of the RNA-guided CRISPR-nuclease complex. For instance, in case the CRISPR protein is S. pyogenes Cas9, the PAM sequence preferably is 5′-NGG-3′, wherein N can be any one of T, G, A or C. The skilled person can straightforwardly engineer a crRNA to target any desired sequence, preferably by engineering the sequence to be at least partly complementary to any desired protospacer sequence, in order to hybridize thereto. Preferably, the complementarity between part of a crRNA sequence and its corresponding protospacer sequence, when optimally aligned using a suitable alignment algorithm, is at least 70%, 80%, 85%, 90%, 91%, 92%, 93%, 94%, 95%, 96%, 97%, 98% or 100%. The part of the crRNA sequence that is complementary to the protospacer sequence may be at least about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. Preferably the part of the crRNA that is complementary to the protospacer sequence is 15, 16, 17, 18, 19, 20, 21, 22, 23, 24 or 25 nucleotides in length, preferably 20 or 21 nucleotides, preferably 20 nucleotides. Molecules or sequences suitable as crRNA and tracrRNA are well known in the art (see e.g., WO2013142578 and Jinek et al., Science (2012) 337, 816-821). Preferably, the crRNA and tracrRNA are linked together to form a single guide (sg)RNA. The crRNA and tracrRNA can be linked, preferably covalently linked, using any conventional method known in the art. Covalent linkage of the crRNA and tracrRNA is e.g. described in Jinek et al. (supra) and WO13/176772, which are incorporated herein by reference. The crRNA and tracrRNA can be covalently linked using e.g. linker nucleotides or via direct covalent linkage of the 3′ end of the crRNA and the 5′ end of the tracrRNA.

A “CRISPR-nuclease complex” or “gRNA-CAS complex” is to be understood herein as a CAS protein, also named a CRISPR-nuclease, which is complexed or hybridized to a guide RNA, wherein the guide RNA may be a crRNA and/or a tracrRNA, or a sgRNA.

A “CRISPR-nickase” is a variant of the CRISPR-nuclease wherein one of the nuclease domains is mutated such that it is no longer functional (i.e., the nuclease activity is absent). An example is a SpCas9 variant having either a D10A or H840A mutation.

A “dead CRISPR-nuclease” is a variant of the CRISPR-nuclease, comprising modifications such that none of the nuclease domains shows cleavage activity. An example is a SpCas9 variant having both an D10A and H840A mutation. The term “endogenous” as used in the context of the present invention in combination with a protein or nucleic acid means that said protein or nucleic acid is contained within the plant, i.e. is present in its natural environment. Often an endogenous gene will be present in its normal genetic context in the plant.

An “endonuclease”, also indicated herein as “nuclease” or “nuclease element”, is an enzyme that hydrolyses the phosphodiester bond in a nucleic acid chain. A nuclease or endonuclease is understood herein as a protein being capable of hydrolysing both strands of the duplex, preferably at the same time, to introduce a double strand break in the DNA.

A “nickase” is understood herein as a protein, optionally being an endonuclease with one inactive domain and one active domain, that hydrolyses only one strand of a duplex DNA to produce DNA molecules that are “nicked” rather than cleaved.

The term “Exemplary” means “serving as an example, instance, or illustration,” and should not be construed as excluding other configurations disclosed herein.

“Expression of a gene” refers to the process wherein a DNA region which is operably linked to appropriate regulatory regions, particularly a promoter, is transcribed into an RNA, which is biologically active, e.g. which is capable of being translated into a biologically active protein or peptide, or e.g. a regulatory non-coding RNA.

The term “gene” means a nucleic acid fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence, a coding region and a 3′ non-translated sequence (3′ end) comprising a polyadenylation site.

A “guide element” or “guide” is to be understood herein as an element that directs a nuclease element to a specific sequence in a (double-stranded) nucleic acid molecule. A guide element comprises a guide sequence for recognizing and targeting a site-directed nuclease to a specific sequence in a nucleic acid molecule, preferably in a genome. The guide element is preferably at least one of: (i) an oligonucleotide, preferably an RNA and/or DNA oligonucleotide, capable of hybridizing or complexing with a nuclease (element) to form a site-directed nuclease; and (ii) a polypeptide, protein or protein domain that is part of a site-directed nuclease. The polypeptide, protein or protein domain may be covalently bound, fused to, or complexed with a nuclease (element) to form a site-directed nuclease.

A “guide RNA” or “gRNA” is understood herein as guide element that is an RNA molecule comprising a guide sequence for targeting the gRNA-CAS complex to the protospacer sequence that is preferably near, at or within the sequence of interest in the nucleic acid molecule, and may be a sgRNA or the combination of a crRNA and a tracrRNA (e.g. for Cas9) or a crRNA only (e.g. in case of Cpf1). Optionally, more than one type of guide RNA may be used in the same experiment, for example aimed at two or more different sequences of interest, or even aimed at the same sequence of interest.

A “guide sequence” is to be understood herein as a part of a guide element that recognizes, binds and/or hybridizes to a specific site in an RNA or DNA molecule. The guide sequence may be a nucleotide sequence in case the site-specific endonuclease is a RNA or DNA guided endonuclease such as in a gRNA-CAS complex, or may be an amino acid sequence in case the site-specific endonuclease is a protein guided endonuclease, such as a TALEN, ZFN or meganuclease. In the context of a gRNA-CAS complex, “guide sequence” is further to be understood herein as the section of the sgRNA or crRNA, which is required for targeting a gRNA-CAS complex to a specific site in a duplex DNA.

“Plant” refers to either the whole plant or to parts of a plant, such as tissue or organs (e.g. pollen, seeds, gametes, roots, leaves, flowers, flower buds, anthers, fruit, etc.) obtainable from the plant, as well as derivatives of any of these and progeny derived from such a plant by selfing or crossing. Non-limiting examples are barley, cassava, cotton, groundnuts or peanuts, maize, millet, oil palm fruit, potatoes, pulses, rapeseed or canola, rice, rye, sorghum, soybeans, sugar cane, sugar beets, sunflower, wheat, okra, allium, celery, asparagus, wax gourd, beet, brassica including vegetable brassica, bell pepper, hot pepper, endive, chicory, melon including watermelon, cucumber, gherkin, zucchini, pumpkin, artichoke, carrot, rocket, fennel, lettuce, bottle gourd, ridge gourd, sponge gourd, bitter gourd, parsnip, parsley, common bean, bladder cherry, tomatillo, radish, eggplant, tomato including tomato rootstock, pepino, spinach, snake gourd, corn salad, pea including chick pea, soybean, broad bean, sweet corn, hemp, hop, berries, dandelion and ornamentals.

The terms “protein” or “polypeptide” are used interchangeably and refer to molecules consisting of a chain of amino acids, without reference to a specific mode of action, size, 3 dimensional structure or origin. A “fragment” or “portion” of a protein may thus still be referred to as a “protein”. An “isolated protein” is used to refer to a protein which is no longer in its natural environment, for example in vitro or in a recombinant bacterial or plant host cell.

The terms “sequence identity”, “homology” and the like are used interchangeably herein. Sequence identity is herein defined as a relationship between two or more amino acid (polypeptide or protein) sequences or two or more nucleotide (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods. The percentage sequence identity/similarity can be determined over the full length of the sequence. As used herein “sequence identity” refers to the extent to which two optimally aligned polynucleotide or peptide sequences are invariant throughout a window of alignment of components, e.g., nucleotides or amino acids. An “identity fraction” for aligned segments of a test sequence and a reference sequence is the number of identical components which are shared by the two aligned sequences divided by the total number of components in reference sequence segment, i.e. the entire reference sequence or a smaller defined part of the reference sequence. “Percent identity” is the identity fraction times 100. “Sequence identity” and “sequence similarity” can be determined by alignment of two peptide or two nucleotide sequences using global or local alignment algorithms, depending on the length of the two sequences. Sequences of similar lengths are preferably aligned using a global alignment algorithms (e.g. Needleman Wunsch) which aligns the sequences optimally over the entire length, while sequences of substantially different lengths are preferably aligned using a local alignment algorithm (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined herein). The percent of sequence identity is preferably determined using the “BESTFIT” or “GAP” program of the Sequence Analysis Software Package™ (Version 10; Genetics Computer Group, Inc., Madison, Wis.). GAP uses the Needleman and Wunsch global alignment algorithm (Needleman and Wunsch, Journal of Molecular Biology 48:443-453, 1970) to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blossum62 for proteins and DNAFull for DNA). gap extension penalty is 0.5; default scoring matrices are Blossum62 for proteins and DNAFull for DNA). “BESTFIT” performs an optimal alignment of the best segment of similarity between two sequences and inserts gaps to maximize the number of matches using the local homology algorithm of Smith and Waterman (Smith and Waterman, Advances in Applied Mathematics, 2:482-489, 1981, Smith et al., Nucleic Acids Research 11:2205-2220, 1983). When sequences have a substantially different overall lengths, local alignments, such as those using the Smith Waterman algorithm, are preferred. Useful methods for determining sequence identity are also disclosed in Guide to Huge Computers, Martin J. Bishop, ed., Academic Press, San Diego, 1994, and Carillo, H., and Lipton, D., Applied Math (1988) 48:1073. More particularly, preferred computer programs for determining sequence identity include the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; Altschul et al., J. Mol. Biol. 215:403-410 (1990); version 2.0 or higher of BLAST programs allows the introduction of gaps (deletions and insertions) into alignments; for peptide sequence BLASTX can be used to determine sequence identity; and, for polynucleotide sequence BLASTN can be used to determine sequence identity. Alternatively percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

A “3′ non-translated sequence” or “3′ UTR” (also often referred to as 3′ untranslated region, or 3′end) refers to the nucleic acid sequence found downstream of the coding sequence of a gene, which comprises for example a transcription termination site and (in most, but not all eukaryotic mRNAs) a polyadenylation signal (such as e.g. AAUAAA or variants thereof). After termination of transcription, the mRNA transcript may be cleaved downstream of the polyadenylation signal and a poly (A) tail may be added, which is involved in the transport of the mRNA to the cytoplasm (where translation takes place).

A “nucleic acid” or “polynucleotide” as used herein may include any polymer or oligomer of pyrimidine and purine bases, preferably cytosine, thymine, and uracil, and adenine and guanine, respectively (See Albert L. Lehninger, Principles of Biochemistry, at 793-800 (Worth Pub. 1982) which is herein incorporated by reference in its entirety for all purposes). The present invention contemplates any deoxyribonucleotide, ribonucleotide or nucleic acid component, and any chemical variants thereof, such as methylated, hydroxymethylated or glycosylated forms of these bases, and the like. The polymers or oligomers may be heterogeneous or homogenous in composition, and may be isolated from naturally occurring sources or may be artificially or synthetically produced. In addition, the nucleic acids may be DNA (optionally cDNA) or RNA, or a mixture thereof, and may exist permanently or transitionally in single-stranded or double-stranded form, including homoduplex, heteroduplex, and hybrid states. An “isolated nucleic acid” is used to refer to a nucleic acid which is no longer in its natural environment, for example in vitro or in a recombinant bacterial or plant cell.

The terms “nucleic acid construct”, “nucleic acid vector”, “vector” and “expression construct” are used interchangeably herein and is herein defined as a man-made nucleic acid molecule resulting from the use of recombinant DNA technology. The terms “nucleic acid construct” and “nucleic acid vector” therefore does not include naturally occurring nucleic acid molecules although a nucleic acid construct may comprise (parts of) naturally occurring nucleic acid molecules.

The vector backbone may for example be a binary or superbinary vector (see e.g. U.S. Pat. Nos. 5,591,616, 20,021,38879 and WO 95/06722), a co-integrate vector or a T-DNA vector, as known in the art and as described elsewhere herein, into which a chimeric gene is integrated or, if a suitable transcription regulatory sequence is already present, only a desired nucleic acid sequence (e.g. a coding sequence, an antisense or an inverted repeat sequence) is integrated downstream of the transcription regulatory sequence. Vectors can comprise further genetic elements to facilitate their use in molecular cloning, such as e.g. selectable markers, multiple cloning sites and the like.

The term “operably linked” refers to a linkage of polynucleotide elements in a functional relationship. A nucleic acid is “operably linked” when it is placed into a functional relationship with another nucleic acid sequence. For instance, a promoter, or rather a transcription regulatory sequence, is operably linked to a coding sequence if it affects the transcription of the coding sequence. Operably linked may mean that the DNA sequences being linked are contiguous.

“Plant cell(s)” include protoplasts, gametes, suspension cultures, microspores, pollen grains, etc., either in isolation or within a tissue, organ or organism. The plant cell can e.g. be part of a multicellular structure, such as a callus, meristem, plant organ or an explant.

“Plant hormone”, “plant growth hormone”, “plant growth regulator” or “phytohormone” is a chemical that influences the growth and/or development of plant cells and tissues. Plant growth regulators comprise chemicals from the following five groups: auxins, cytokinins, gibberellins, abscisic acid (ABA) and ethylene. In addition to the five main groups, two other classes of chemical are often regarded as plant growth regulators: brassinosteroids and polyamines.

“Promoter” refers to a nucleic acid fragment that functions to control the transcription of one or more nucleic acids. A promoter fragment is preferably located upstream (5′) with respect to the direction of transcription of the transcription initiation site of the gene, and is structurally identified by the presence of a binding site for DNA-dependent RNA polymerase, transcription initiation site(s) and can further comprise any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive promoter” is a promoter that is active in most tissues under most physiological and developmental conditions. An “inducible promoter” is a promoter that is physiologically (e.g. by external application of certain compounds) or developmentally regulated. A “tissue specific promoter” is only active in specific types of tissues or cells. Optionally the term “promoter” may also include the 5′ UTR region (5′ Untranslated Region) (e.g. the promoter may herein include one or more parts upstream of the translation initiation codon of transcribed region, as this region may have a role in regulating transcription and/or translation).

The “protospacer sequence” is the sequence that is recognized or can be hybridized to a guide sequence within a guide element, preferably within a guide RNA, more specifically the crRNA or, in case of a sgRNA, the crRNA part of the guide RNA, and is located in, at or near the target nucleic acid fragment.

The term “regeneration” is herein defined as the formation of a new tissue and/or a new organ from a single plant cell, a callus, an explant, a tissue or from an organ. Regeneration may include the formation of a new plant from a single plant cell or from e.g. a callus, an explant, a tissue or an organ. The regeneration process can occur directly from parental tissues or indirectly, e.g. via the formation of a callus. The regeneration pathway can be somatic embryogenesis or organogenesis. Somatic embryogenesis is understood herein as the formation of somatic embryos, which can be grown to regenerate whole plants. Organogenesis is understood herein as the formation of new organs from (undifferentiated) cells. Organogenesis may be at least one of meristem formation, adventitious shoot formation, inflorescence formation, root formation, elongation of adventitious shoots and (subsequent) the formation of a complete plant. Preferably, regeneration is at least one of shoot regeneration and (ectopic) apical meristem formation. Shoot regeneration as defined herein is de novo shoot formation. For example, regeneration can be the regeneration of a(n) (inflorescence) shoot from a(n) (elongated) hypocotyl (explant).

The term “sequence of interest” includes, but is not limited to, any genetic sequence preferably present within a cell, such as, for example a gene, part of a gene, or a non-coding sequence within or adjacent to a gene. The sequence of interest may be present in a chromosome, an episome, an organellar genome such as mitochondrial or chloroplast genome or genetic material that can exist independently to the main body of genetic material such as an infecting viral genome, plasmids, episomes, transposons for example. A sequence of interest may be within the coding sequence of a gene, within transcribed non-coding sequence such as, for example, leader sequences, trailer sequence or introns. Said sequence of interest may be present in a double or a single strand nucleic acid molecule. The nucleic acid sequence is preferably present in a double-stranded nucleic acid molecule. The sequence of interest may be any sequence within a nucleic acid, e.g., a gene, gene complex, locus, pseudogene, regulatory region, highly repetitive region, polymorphic region, or portion thereof. The sequence of interest may also be a region comprising genetic or epigenetic variations indicative for a phenotype or disease. Preferably, the sequence of interest is a small or longer contiguous stretch of nucleotides (i.e. a polynucleotide) of duplex DNA, wherein said duplex DNA further comprises a sequence complementary to the target sequence in the complementary strand of said duplex DNA.

“Shoot organogenesis” is the regeneration pathway by which cells form a de novo shoot apical meristem that develops into a shoot with leaf primordia and leaves. As there is only one apical meristem, this is a unipolar structure, and roots are not formed at this stage. The vascular system of the shoot is often connected to the parent tissue. Only after the shoots have fully formed and elongated, and are taken off, can the formation of roots be induced, preferably in a separate root induction step on a different culture medium (Thorpe, TA (1993) In vitro Organogenesis and Somatic Embryogenesis: Physiological and Biochemical Aspects. In: Roubelakis-Angelakis K. A., Van Thanh K. T. (eds) Morphogenesis in Plants. NATO ASI Series (Series A: Life Sciences), Vol. 253. Springer, Boston, MA). Shoot organogenesis may occur spontaneously, i.e. without the external addition of any plant growth regulators (PGRs). Shoot organogenesis may be induced by plant growth regulators, usually cytokinins alone in different concentrations or in combination with an auxin, wherein preferably the cytokinins remain a constituent of the culture media until the new shoot apical meristems and the shoots have been formed and are sufficiently elongated, e.g. to take them off. Preferably, for the induction of shoot formation, the concentration of cytokinins exceeds the concentration of auxins.

“Similar conditions” for culturing the plant/plant cells means among other things the use of a similar temperature, humidity, nutrition and light conditions, and similar irrigation and day/night rhythm.

“Site-directed (endo) nuclease”, also indicated herein as a “site-specific nuclease” is understood herein as nucleic acid-protein complex or a protein (dimer) that comprises both a guide element as defined herein and a nuclease or nuclease element as defined herein, wherein said guide element targets the nuclease element to a target sequence preferably present in the genome, preferably a protospacer sequence. Preferably, the site-directed nuclease is a programmable nuclease such as, but not limited to, zinc finger nuclease (ZFN), transcription activator-like endonucleases (TALEN), meganuclease, clustered regularly interspaced short palindromic repeats (CRISPR)-nuclease complex and Argonaute. The site-directed nuclease may have endonuclease activity, preferably being capable of introducing a double strand break in duplex DNA, or may be modified to show reduced endonuclease activity, e.g. rendering nickase, preferably being capable of introducing a single strand break in duplex DNA, or abolished nuclease activity, rendering a dead nuclease.

“Targeted mutagenesis” is mutagenesis that can be designed to alter a specific nucleotides or nucleic acid sequence, such as but not limited to, oligo-directed mutagenesis, RNA-guided endonucleases (e.g. the CRISPR-technology), TALENs or Zinc finger technology.

The term “wild type” as used in the context of the present invention in combination with a protein or nucleic acid means that said protein or nucleic acid consists of an amino acid or nucleotide sequence, respectively, that occurs as a whole in nature and can be isolated from organisms in nature as such, e.g. is not the result of modification techniques such as targeted or random mutagenesis or the like. A wild type protein is expressed in at least a particular developmental stage under particular environmental conditions, e.g. as it occurs in nature.

### DETAILED DESCRIPTION

The inventors have discovered a method for increasing the number of genetically modified plant shoots after vector-mediated transfection of at least one element of a site-directed nuclease in planta. These modified shoots preferably also comprise a high percentage of modified cells. In a first aspect, the method pertains to a method for producing a shoot of a plant, wherein the shoot comprises one or more cells having a genetic modification. The method of the invention preferably comprises the steps of:
i) providing a plant;
ii) introducing in a cell of the plant a vector expressing at least one of a guide element and a nuclease element of a site-directed nuclease;
iii) removing the apical meristem of the plant; and
iv) regenerating a shoot at a second location in the plant, wherein the shoot comprises one or more cells having a genetic modification.

Preferably, the order of steps in the method of the invention is step i), ii), iii), and iv). Alternatively, the order of steps can be step i), iii), ii) and iv). The method of the invention as detailed herein may also be considered at least one of e.g.
a method for producing a plant having one or more genetically modified cells;
a method for producing a transgenic plant; and
a method for genetically modifying a shoot of a plant.

Preferably, the method of the invention comprises the steps of:
i) providing a plant, wherein the plant expresses a nuclease element of a site-directed nuclease;
ii) introducing in a cell of the plant a vector expressing a guide element of said site-directed nuclease;
iii) removing the apical meristem of the plant; and
iv) regenerating a shoot at a second location in the plant, wherein the shoot comprises one or more cells having a genetic modification.

Alternatively, the method of the invention may comprise the steps of:
i) providing a plant;
ii) introducing in a cell of the plant a vector expressing a site-directed nuclease;
iii) removing the apical meristem of the plant; and
iv) regenerating a shoot at a second location in the plant, wherein the shoot comprises one or more cells having a genetic modification.

The genetic modification (or “heritable modification”) is preferably in a sequence of interest. The sequence of interest is preferably part of, or operably linked to, a gene of interest. A gene of interest preferably refers to any genomic or episomal DNA sequence in a cell that one desired to target for cleavage and possible alteration. The site-directed nuclease of the method of the invention is designed to target to a sequence at or near said sequence of interest in order to induce a genetic modification in said sequence of interest. Hence, the genetic modification of the method of the invention is a targeted genetic modification. In some embodiments, the gene can encode a protein. In some embodiments, the gene encodes a non-coding RNA. In some embodiments, the portion of the gene targeted is a promoter, enhancer, or coding or non-coding sequence.

### Step i)—Providing a Plant

Preferably, the first step of the method of the invention is the provision of a plant. The provided plant is preferably capable of regeneration, preferably capable of regenerating one or more shoots. Preferably, the plant is not a recalcitrant plant. Preferably, the plant in step i) is a regenerative plant. Preferably, a regenerative plant is capable of forming de novo shoots on a multicellular tissue. A regenerative plant provided in step i) is preferably capable of regeneration under normal growth conditions, optionally in the presence of externally supplied growth regulators such as auxins and/or cytokines. Such normal growth and/or optimal conditions at least comprise suitable nutrient supply, optionally supplemented with plant hormones. Such conditions may further encompass a suitable and/or optimal temperature and/or light/dark regime.

Preferably, the provided plant in step i) is capable of regenerating shoots after wounding in vivo. Preferably, the provided plant in step i) is capable of regenerating shoots after decapitation in vivo. Decapitation is understood herein as the removal of a preformed meristem, apical or axillary. Although within species both recalcitrant and regenerative cultivars, varieties and/or accessions may exist, in general, species of the family Solonaceae such as, but not limited to, Solanum tuberosum, Solanum lycopersicum and Nicotiana tabacum, have long been reported to be regenerative species (Roest and Gilissen, Acta Bot. Neerl. 1998; 38 (1): p1-23). At present, understanding of the mechanism of shoot regeneration has resulted in effective protocols for shoot regeneration of many different species including more recalcitrant species (for review see e.g. Shin et al. Journal of Experimental Botany, 2020; 71 (1):63-72).

Preferably the provided plant is a seedling, a vegetative plant, a budding plant, a flowering plant or a ripening plant. Preferably, the provided plant is a seedling.

The provided plant can be a dicotylenous plant. The provided plant may be a naturally occurring regenerative plant, i.e. a plant that has a natural ability to regenerate. Alternatively, the provided plant may be genetically modified to induce or increase the regeneration potential. Examples of such plants include, but are not limited to, plants disclosed in WO2019/211296 and WO2019/193143, which are incorporated herein by reference. As a non-limiting example, the provided regenerative plant can be a plant modified to express a histidine kinase selected from the group consisting of CHK4, CHK2 and CHK3. In addition or alternatively, the provided regenerative plant is a plant modified to, preferably transiently, express at least one of WOX5 and a PLT protein, preferably at least one of WOX5 and PLT1.

The plant provided in step i) may be a cultivar or a wild type plant. Optionally, the plant is a genetically modified plant. The method of the invention may comprise the use of a site-directed (programmable) nuclease that functions as a non-covalently assembled complex, e.g. the site-directed nuclease may consists of a nuclease element, such as a CAS protein, complexed with a guide element, such as a guide RNA. In this case, the provided plant may be genetically modified to (stably) express at least one of the guide element and the nuclease element. Optionally, the provided plant has been modified to express said nuclease element in at least part of the plant. Optionally, the provided plant has been modified to stably express a guide element or a nuclease element of a site-directed nuclease. Optionally, the plant has been modified to express said nuclease element or guide element after activation of an inducible promoter. Said nuclease element preferably is a CRISPR-nuclease or an Argonaute and said guide element preferably is a guide RNA. The provided plant may express said guide element or nuclease element in all, or substantially all, cells. Optionally, the provided plant has been modified to express at least one of a CRISPR-nuclease and a guide-RNA. Optionally, the plant is modified to express a CRISPR-nuclease, wherein the CRISPR-nuclease is a CRISPR-nuclease as defined herein below. Preferably, the CRISPR-nuclease is at least one of Cas9, Cpf1 and Mad7. Optionally, the provided plant has been modified to express an Argonaute.

In step i), more than one plant may be provided. Optionally, at least 2, 5, 10, 15, 20, 50, 100, 200, 500, 1000, 5000 or more plants are provided in step i). These plants may be simultaneously, or essentially simultaneously subjected to the method of the invention as specified herein.

### Step ii)—Introducing a Vector

In step ii) of the method of the invention, a plant cell of the provided plant is exposed to a vector, resulting in the introduction of the vector into a cell of the provided plant. The vector encodes at least one element of a site-directed nuclease.

In case the method of the invention makes use of a site-directed (programmable) nuclease comprising a nuclease element that is covalently linked to a guide element,, e.g. a meganuclease, ZFN or TALEN, the vector preferably encodes for both the nuclease element and the guide element. In case the method of the invention makes use of a site-directed (programmable) nuclease that functions as a non-covalently assembled complex, such as a CAS protein complexed with a guide RNA, the plant may be genetically modified to (stably) express at least one of the elements of the complex, and the vector may comprise the other element of the complex. For instance, the plant may be genetically modified to express a nuclease element, such as a CAS protein, and the vector may encode one or more guide elements, such as guide RNAs, for targeting the nuclease element to one or more target sequences. Alternatively, the vector may encode for both the nuclease element, such as a CAS protein, and one or more guide elements, such as one or more guide RNAs. Optionally, in step ii) the plant may be exposed to multiple vectors, wherein at least one of these vectors encodes a nuclease element, such as a CAS protein, and another encodes for a guide element, such as a guide RNA, for targeting the nuclease element to a target sequence.

The vector, preferably a viral vector, may be comprised in a viral particle or an Agrobacterium to initially introduce the vector into a cell of the plant. A virus particle for use in the method of the invention may be produced for instance in Nicotiana benthamiana.

After infection in step ii) of the method of the invention, the vector is expressed from the virus or Agrobacterium in the plant cell. A viral vector may subsequently replicate and infect surrounding plant cells. A preferred Agrobacterium for introducing a vector is Agrobacterium tumefaciens.

The vector may be introduced by agrobacterium or virus particle infiltration, painting, rubbing, brushing, spraying, and dipping. Optionally, a biolistic approach is used, i.e. vector may be introduced by agrobacterium or virus particle bombardment. Preferably, the vector is introduced by agrobacterium painting of leaf material using a swab.

The vector may be introduced in cells of any part of the provided plant. The vector can be introduced in cells of the shoot system and/or in cells of the root system. The vector can be introduced in cells of a root, stem, fruit, leaf, internode, and/or a flower. Preferably, the vector is introduced in cells of a seedling. As a non-limiting example, the vector can be introduced in cells of a true leaf, epicotyl, cotyledon, hypocotyl and/or in cells of the radicle. Preferably, the vector is introduced in cells of a cotyledon. Preferably, the vector is introduced in a cell of the provided plant, preferably in cells of a cotyledon of said plant, wherein the plant is a young seedling consisting of the radicle (embryonic root), the hypocotyl (embryonic shoot), and the cotyledons. Preferably, the vector is introduced in a cell of the provided plant, preferably in cells of a cotyledon of said plant, about 2, 3, 4, 5, 6, 7, 8, 9, 10, 11, 12, 13, 14 days, preferably about 7-10 days, after sowing.

The vector preferably expresses a site-directed nuclease, preferably selected from the group consisting of a CRISPR-nuclease complex or an element thereof, an Argonaute complex or an element thereof, a Zinc finger nuclease, a TALEN and a meganuclease. Preferably, the vector expresses a CRISPR-nuclease complex or an element thereof, wherein preferably the element is at least one of a CRISPR-nuclease and a gRNA. Preferably, the vector expresses both a CRISPR-nuclease and a gRNA.

The provided plant may be exposed to a single vector. Optionally said single vector is PVX or Geminivirus. Optionally said single vector expresses both a CRISPR-nuclease and a guide RNA, optionally a CISPR-nuclease and multiple guide RNAs. Optionally, the provided plant is exposed to 2, 3, 4, 5, 6, 7, 8, 9, 10 or more different vectors. Preferably, the plant is exposed to at least two vectors, wherein one vector expresses a CRISPR-nuclease and another vector expresses a guide RNA, optionally multiple guide RNAs.

A vector for use in the method of the invention preferably comprises a sequence encoding a site-directed nuclease as defined herein, in addition to one or elements for expression of the encoded site-directed nuclease. The vector for use in the method of the invention preferably expresses at least one of a CRISPR-nuclease and a guide RNA (gRNA). The vector of the invention preferably expresses a CRISPR-nuclease, wherein said nuclease may have endonuclease or nickase activity (CRISPR-nickase), or is a dead CRISPR-nuclease. Optionally the CRISRP-nuclease is fused to one or more deaminase domains.

The (nuclease element of) the site-directed nuclease, wherein preferably the nuclease element is a CRISPR-nuclease, may comprise a nuclear localisation signal (NLS) to direct the expressed nuclease element to the nucleus of the plant cell. The NLS may be located at the C-terminus and/or at the N-terminus of the (nuclease element of the) site-directed nuclease. Any known nuclear localisation signal would be suitable for use in the invention. Preferred nuclear localisation signals include, but are not limited to the NLS of the SV40 Large T-antigen MEDPTMAPKKKRKV (SEQ ID NO: 1), the monopartite NLS PKKKRKV (SEQ ID NO: 2) and the NLS of nucleoplasmin KRPAATKKAGQAKKKK (SEQ ID NO: 3). The (nuclease element of the) site-directed nuclease may comprise two or more nuclear localisation signals, e.g. one or more at the N-terminus and one or more at the C-terminus. The (nuclease element of the) site-directed nuclease may comprise two or more different nuclear localisation signals, e.g. the NLS at the C-terminus may differ from the NLS at the N-terminus.

The nuclease element for use in the invention may be any CRISPR-nuclease as defined herein. Preferably, the nuclease element is a Type II CRISPR-nuclease, preferably a Type II CRISPR-nuclease, e.g., Cas9 (e.g., the protein of SEQ ID NO: 4, encoded by SEQ ID NO: 5, or the protein of SEQ ID NO: 6) or a Type V CRISPR-nuclease, preferably a Type V CRISPR-nuclease, e.g. Cpf1 (e.g., the protein of SEQ ID NO: 7, encoded by SEQ ID NO: 8) or Mad7 (e.g. the protein of SEQ ID NO: 9 or 10), or a protein derived thereof, having preferably at least about 70%, 80%, 90%, 91%, 92%, 93%, 94%, 95%, 96%, 97%, 98%, or 99% sequence identity to said protein over its whole length.

Preferably, the nuclease element of the site-directed nuclease is a Type II CRISPR-nuclease, preferably a Cas9 nuclease.

The skilled person knows how to find and prepare a vector comprising a sequence encoding the (nuclease element of the) site-directed nuclease, such as a sequence encoding a CRISPR-nuclease. In the prior art, numerous reports are available on its design and use. See for example the review by Haeussler et al (J Genet Genomics. (2016)43(5):239-50. doi: 10.1016/j.jgg.2016.04.008.) on the design of guide RNA and its combined use with a CAS-protein (originally obtained from S. pyogenes), or the review by Lee et al. (Plant Biotechnology Journal (2016) 14(2) 448-462).

Optionally, the nuclease element of the site-directed nuclease is a CRISPR-nuclease being either a nickase or (endo) nuclease.

The nuclease element expressed by the vector may comprise or consist of a whole type II or type V CRISPR-nuclease or variant or functional fragment thereof. Optionally, such fragment binds the guide RNA, but e.g. may lack one or more residues required for nuclease activity.

Preferably, the nuclease element of the site-directed nuclease is a Cas9 protein. The Cas9 protein may be derived from the bacteria Streptococcus pyogenes (SpCas9; NCBI Reference Sequence NC_017053.1; UniProtKB-Q99ZW2), Geobacillus thermodenitrificans (UniProtKB-A0A178TEJ9), Corynebacterium ulcerous (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquisl (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); or Neisseria meningitidis (NCBI Ref: YP_002342100.1). Encompassed are Cas9 variants from these, having an inactivated HNH or RuvC domain homologues to SpCas9,, e.g. the SpCas9_D10A or SpCas9_H840A, or a Cas9 having equivalent substitutions at positions corresponding to D10 or H840 in the SpCas9 protein, rendering a nickase.

The nuclease element of the site-directed nuclease may be, or may be derived from, Cpf1, e.g. Cpf1 from Acidaminococcus sp; UniProtKB-U2UMQ6. The variant may be a Cpf1-nickase having an inactivated RuvC or NUC domain, wherein the RuvC or NUC domain has no nuclease activity anymore. The skilled person is well aware of techniques available in the art such as site-directed mutagenesis, PCR-mediated mutagenesis, and total gene synthesis that allow for inactivated nucleases such as inactivated RuvC or NUC domains. An example of a Cpf1 nickase with an inactive NUC domain is Cpf1 R1226A (see Gao et al. Cell Research (2016) 26:901-913, Yamano et al. Cell (2016) 165 (4): 949-962). In this variant, there is an arginine to alanine (R1226A) conversion in the NUC-domain, which inactivates the NUC-domain.

The nuclease element of the site-directed nuclease may be, or may be derived from, CRISPR-Casϕ, a nuclease that is about half the size of Cas9. CRISPR-Casϕ uses a single crRNA for targeting and cleaving the nucleic acid as is described e.g. in Pausch et al (CRISPR-Casϕ from huge phages is a hypercompact genome editor, Science (2020); 369(6501):333-337).

An active, partly inactive or dead site-directed nuclease, preferably an active, partly inactive or dead CRISPR-nuclease complex may serve to guide a fused functional domain to a specific site in the DNA as determined by the guide element, preferably a guide RNA. Hence, the (nuclease element of the) site-directed nuclease may be fused to a functional domain. Optionally, such functional domain is for epigenetic modification, for example a histone modification domain. The domains for epigenetic modification can be selected from the group consisting of a methyltransferase, a demethylase, a deacetylase, a methylase, a deacetylase, a deoxygenase, a glycosylase and an acetylase (Cano-Rodriguez et al, Curr Genet Med Rep (2016) 4:170-179). The methyltransferase may be selected from the group consisting of G9a, Suv39h1, DNMT3, PRDM9 and Dot1L. The demethylase may be LSD1. The deacetylase may be SIRT6 or SIRT3. The methylase may be at least one of KYP, TgSET8 and NUE. The deacetylase may be selected from the group consisting of HDAC8, RPD3, Sir2a and Sin3a. The deoxygenase may be at least one of TET1, TET2 and TET3, preferably TET1cd (Gallego-Bartolomé J et al, Proc Natl Acad Sci USA. (2018); 115(9):E2125-E2134). The glycosylase may be TDG. The acetylase may be p300.

Optionally, the functional domain is a deaminase, or functional fragment thereof, selected from the group consisting of an apolipoprotein B mRNA-editing complex (APOBEC) family deaminase, an activation-induced cytosine deaminase (AID), an ACF1/ASE deaminase, an adenine deaminase, and an ADAT family deaminase. Alternatively or in addition, the deaminase or functional fragment thereof may be ADAR1 or ADAR2, or a variant thereof.

The apolipoprotein B mRNA-editing complex (APOBEC) family of cytosine deaminase enzymes encompasses eleven proteins that serve to initiate mutagenesis in a controlled and beneficial manner. Preferably, the APOBEC deaminase is selected from the group consisting of APOBEC1, APOBEC2, APOBEC3A, APOBEC3B, APOBEC3C, APOBEC3D, APOBEC3F, APOBEC3G, APOBEC3H, APOBEC4 and Activation-induced (cytidine) deaminase. Preferably, the cytosine deaminase of the APOBEC family is activation-induced cytosine (or cytidine) deaminase (AID) or apolipoprotein B editing complex 3 (APOBEC3). Preferably, the deaminase domain fused to the CRISPR-nuclease an APOBEC1 family deaminase.

Another exemplary suitable type of deaminase domain that may be fused to the nuclease element of the site-directed nuclease, preferably to the CRISPR-nuclease, is an adenine or adenosine deaminase, for example an ADAT family of adenine deaminase. Further, the adenine deaminase may be TadA or a variant thereof, preferably as described in Gaudelli et al., 2017 (Gaudelli et al. 2017 Nature 551:464-471). Further, the nuclease element, preferably the CRISPR-nuclease, may be fused to an adenine deaminase domain, e.g. derived from ADAR1 or ADAR2. The deaminase domain of the present invention may comprise or consist of a whole deaminase protein or a fragment thereof which has catalytic activity. Preferably, the deaminase domain has deaminase activity. Optionally, the nuclease element, preferably the CRISPR-nuclease, is further fused to an UDG inhibitor (UGI) domain.

The nuclease element of the site-directed nuclease, preferably the CRISPR-nuclease, may be fused to a reverse transcriptase, e.g. as described in Anzalone AV, et al (Nature (2019), 576, pages149-157), preferably for use in prime-editing.

The vector for use in the method of the invention may comprise two or more sequences encoding a nuclease element of site-directed nucleases. Optionally, the vector may encode two times or more often the same nuclease elements. Alternatively or in addition, the vector may encode for two or more different nuclease elements, such as, but not limited to a CRISPR-Cas9 and a CRISPR-Cpf1.

The nuclease element of the site-directed nuclease may be an Argonaute protein. Argonaute (Ago) proteins bind small RNA or DNA guides, which provide base-pairing specificity for recognition and cleavage of complementary nucleic acid targets (Kaya et al. PNAS 2016 Apr. 12; 113 (15): 4057-4062). Hence the plant proved in step i) may express an Argonaute protein and in step ii) a guide is provided to direct the Argonaute protein to a specific location in the genome of a plant cell to achieve a targeted genetic modification. Alternatively, the guide and Argonaute protein are expressed from one or more vectors in step ii). The Argonaute proteins may cleave DNA, in a process known as DNA interference as described e.g. in Kuzmenko et al (Nature (2020), 587, 632-637). Optionally, the Argonaute protein is complexed with or fused one or more functional domains or proteins, preferably at least one of a helicase and topoisomerase domain, preferably to unwind the genomic DNA.

Optionally, the site-directed nuclease of the method of the invention is a protein that comprises a domain that is designed to recognize and target the nuclease (element) to a specific location in the genome, such as e.g. TALEN comprising a TAL effector DNA-binding domain fused to a DNA cleavage domain, ZFN comprising a zinc finger DNA-binding domain fused to a DNA-cleavage domain, or meganuclease such as I-Scel, I-Crel or I-Dmol. Optionally, the site-directed nuclease is a fusion protein comprising both the nuclease element of the method of the invention in the form of a nuclease domain of said fusion protein, and a guide element of the method of the invention indicated in the form of a guide domain, e.g. zinc finger motifs or TAL effectors, for guidance of the fusion protein to the target sequence to be edited. Optionally, said fusion protein is active, i.e. is capable of inducing a DSB, as a dimer. The expressed site-directed nuclease may be designed to target to a specific location in the genome of a plant cell to achieve a targeted genetic modification. Said target site is preferably near, at or within a sequence of interest in the genome of the plant cell. The vector of the method of the invention may encode both monomers of said dimer. Alternatively, each monomer of the fusion protein is encoded on a separate vector.

In case the nuclease element of the site-directed nuclease is a nuclease that requires complexing with a guide for guidance of the nuclease element to a target sequence to be edited, such as for CRISPR-nuclease or Argonaute, the vector for use in the method of the invention preferably expresses a guide element. The guide element preferably is a guide RNA. The same vector may also express a nuclease element of the site-directed nuclease, preferably a CRISPR-nuclease. Alternatively, the guide element and nuclease element of the site-directed nuclease, preferably a guide RNA and a CRISPR-nuclease, are expressed from separate vectors. Alternatively, the nuclease element of the site-directed nuclease, preferably a CRISPR-nuclease, may already be expressed in the provided plant.

The expressed guide element may guide the nuclease element of the site-directed nuclease to a specific location in the genome of a plant cell to achieve a targeted genetic modification. The guide element directs the complex to a defined target site in a double-stranded nucleic acid molecule, also named the protospacer sequence. The guide element comprises a sequence for targeting the site-directed nuclease complex to a protospacer sequence that is preferably near, at or within a sequence of interest in the genome of the plant cell.

In case the site-directed nuclease is a CRISPR-endonuclease complex, the guide element may be a guide RNA that is a single guide (sg)RNA molecule or the combination of a crRNA and a tracrRNA (e.g. for Cas9) as separate molecules or a crRNA molecule only (e.g. in case of Cpf1 and Casϕ). Optionally, the guide RNA is a single guide (sg)RNA (e.g. for Cas9) or a crRNA only (e.g. in case of Cpf1 and Casϕ). The CRISPR-nuclease complex for use in the method of the invention may thus comprise a guide RNA, wherein the guide RNA comprises a combination of a crRNA and a tracrRNA, and wherein preferably the CRISPR-nuclease is Cas9. The crRNA and tracrRNA are preferably combined into a sgRNA (single guide RNA). Alternatively, the CRISPR-nuclease complex for use in the method of the invention may comprise a guide RNA, wherein the guide RNA comprises a crRNA, and wherein preferably the CRISPR protein is Cpf1 or Casϕ.

The guide element, preferably a guide RNA, for use in a method of the invention may comprise a sequence that can hybridize to or near a sequence of interest, preferably a sequence of interest as defined herein. The guide element, preferably a guide RNA, may comprise a nucleotide sequence that is fully complementary to a sequence in the sequence of interest i.e. the sequence of interest comprises a protospacer sequence. Alternatively or in addition, the guide element, preferably a guide RNA, for use in the method of the invention may comprise a sequence that can hybridize to or near the complement of a sequence of interest.

Optionally, the vector for use in the method of the invention may express more than one guide element, preferably more than one guide RNA, for example to target two or more different sequences of interest, or to target two or more locations of the same sequence of interest.

The vector may express at least 1, 2, 3, 4, 5, 6, 7, 8, 9, 10 or more guide elements or guide RNAs having different seed sequences, i.e. are capable of hybridizing to different protospacer sequences.

The different guide elements may direct the nuclease elements to different locations in the same gene. Alternatively or in addition, the different guide elements may direct the nuclease elements to different genes in the plant cell.

In case the guide element is a guide RNA, optionally the vector does not comprise a spacer sequence located in between the different (pre-)guide RNA sequences, i.e. the different (pre-)RNA guide sequences are located immediately adjacent to each other. Alternatively, there is a sequence of at least 1, 2, 3, 4, 5, 6, 7, 8, 9, 10, 15, 20, 25 or more nucleotides that separate the different (pre-) guide RNA sequences. Optionally, the different (pre-)guide RNA sequences are separated by a cleavable spacer sequence, preferably in case the CRISPR-nuclease is a Cas9 and the guide RNAs are sgRNAs. In case the CRISPR-nuclease is a Cpf1, the (pre-)crRNA guides may not require cleavable spacers as the Cpf1 can process pre-crRNA (Zetsche et al. Nat Biotechnol 2017; 35:31-34).

The cleavable spacer sequence may be any sequence known to the skilled person suitable for cleaving an RNA molecule in a plant cell (e.g. see He et al. J. Genet. Genomics 2017; 44 (9): 469-472). Non-limiting examples of such cleavable spacer sequences include, but are not limited to a spacer having the following sequence: UCGCGGCCGGGUACGUGUUGAGC (SEQ ID NO: 11). The cleavable sequence may be sequence having at least 85%, 90%, 95%, 96%, 97%, 98%, 99% or 100% sequence identity with SEQ ID NO: 11. Alternatively, the cleavable spacer sequence can be any, preferably plant-derived, tRNA sequence or tRNA-like structure. The cleavable tRNA sequence may be a naturally occurring sequence, or may be modified to comprise a cleavable sequence. The tRNA or tRNA-like structure for use in the invention have the ability to function as a cleavable sequence. Any naturally occurring plant tRNA may have the ability to function as a cleavable sequence. The skilled person understands how to straightforwardly obtain the sequence of such plant tRNA or tRNA-like structure.

The naturally occurring tRNA may comprise a recognition site for cleavage by at least one of RNase P and RNase Z. A naturally occurring tRNA may comprise the leader sequence AACAAA, preferably at, or in close vicinity of, the 3′- and/or 5′-end. The leader sequence may enable processing or “cleavage” of the sequence. Preferably, the tRNA comprises the leader sequence AACAAA at, or in close vicinity of, the 5′-end of the tRNA. Suitable tRNA cleavable elements are e.g. described in Xie et al (“Boosting CRISPR/Cas9 multiplex editing capability with the endogenous tRNA-processing system”, 2015, Proc Natl Acad Sci USA; 112(11):3570-5), which is incorporated herein by reference. A preferred cleavable tRNA is tRNA^{Gly}.

Alternatively or in addition, a tRNA or tRNA-like structure as defined herein can be modified to function as a cleavable sequence. The tRNA or tRNA-like structure may be modified to comprise a recognition site for cleavage by at least one of RNase P and RNase Z. Preferably, the tRNA or tRNA-like structure is modified to comprise the leader sequence AACAAA, preferably at, or in close vicinity of, the 3′- and/or 5′-end. Preferably, the tRNA comprises the leader sequence AACAAA at, or in close vicinity of, the 5′-end of the tRNA.

A vector can be used to express the site-directed nuclease, preferably at least one of a CRISPR-nuclease and a guide RNA, in a plant host cell. The vector backbone may for example be a plasmid into which the expression cassette is integrated or, if a suitable transcription regulatory sequence is already present (for example a (inducible) promoter), only a desired nucleotide sequence (e.g. a sequence encoding a guide RNA and/or a CRISPR-nuclease) is integrated downstream of the transcription regulatory sequence.

Suitable promoters include, but are not limited to, viral native coat protein promoters. Such native coat protein promoter for use in the invention may for example be derived from the tobacco rattle virus (as described e.g. in Deng X et al, 2013, Mol Plant-Microbe Interact. doi: 10.1094/MPMI-12-12, 0280-R), the Pea early-browning virus (as described e.g. in MacFarlane S A and Popovich A H (2000),. Virology doi: 10.1006/viro. 1999.0098), any one of the tobacco rattle virus strains TCM, PLB, PSG and PRV (as described e.g. in Goulden M G et al, 1990, Nucleic Acids Res. doi: 10.1093/nar/18.15.4507). The promoter may be a PEBV promoter, a U6 promoter or a pol III promoter.

The vector of the invention may comprise further genetic elements to facilitate their use in molecular cloning, such as e.g. selectable markers, multiple cloning sites and the like. The vector backbone may for example be a binary or superbinary vector (see e.g. U.S. Pat. Nos. 5,591,616, 20,021,38879 and WO 95/06722), a co-integrate vector or a T-DNA vector, as known in the art.

Vectors for use according to the invention are preferably particularly suitable for introducing the expression of at least one element of a site-directed nuclease, preferably a CRISPR-nuclease and/or a guide RNA, into a plant cell. A preferred expression vector is a naked DNA, a DNA complex or a viral vector.

A preferred naked DNA is a linear or circular nucleic acid molecule, e.g. a plasmid. A plasmid refers to a circular double stranded DNA loop into which additional DNA segments can be inserted, such as by standard molecular cloning techniques. A DNA complex can be a DNA molecule coupled to any carrier suitable for delivery of the DNA into the cell. A preferred carrier is selected from the group consisting of a lipoplex, a liposome, a polymersome, a polyplex, PEG, a dendrimer, an inorganic nanoparticle, a virosome and cell-penetrating peptides.

The vector for use in the method of the invention is preferably a viral vector. The viral vector can be a DNA virus or an RNA virus. The viral vector may be, or may be based on, a Tobamovirus, a, Tobravirus, a, Potexvirus a Geminivirus, an Alfamovirus, a Cucumovirus, a Potyvirus, a Tombusvirus, a hordeivirus, or a Nucleorhabdovirus.

The Tobamovirus viral vector may be at least one of a Tobacco Mosaic Virus (TMV) and a Sun Hemp Mosaic Virus (SHMV). The Tobravirus viral vector may be a Tobacco Rattle Virus (TRV). The Potex virus viral vector may be at least one of Potatovirus X (PVX) and the papaya mosaic potexvirus (PapMV). The Geminivirus viral vector may be a Comovirus Cowpea mosaic virus (CPMV). Further examples of suitable Geminivirus viral vectors may include the cabbage leaf curl virus, tomato golden mosaic virus, bean yellow dwarf virus, African cassava mosaic virus, wheat dwarf virus, miscanthus streak mastrevirus, tobacco yellow dwarf virus, tomato yellow leaf curl virus, bean golden mosaic virus, beet curly top virus, maize streak virus, and tomato pseudo-curly top virus. The Alfamovirus may an alfalfa mosaic virus (AMV). The Cucumovirus may be a cucumber mosaic virus (CMV) or a cucumber green mottle mosaic virus (CGMMV). The Potyvirus may be a plum pox virus (PPV). The Tombusvirus may be a tomato bushy stunt virus (TBSV). The hordeivirus may be a barley stripe mosaic virus. The Nucleorhabdovirus may be a Sonchus Yellow Net Virus (SYNV) (see e.g. Hefferon K, Plant Virus Expression Vectors: A Powerhouse for Global Health, Biomedicines. 2017, 5(3):44 and Lico et al, Viral vectors for production of recombinant proteins in plants, J Cell Physiol, 2008; 216(2):366-77).

Preferably, the viral vector is selected from the group consisting of a Tobacco Rattle Virus (TRV), Tobacco Mosaic Virus (TMV), a Sonchus Yellow Net Virus (SYNV) and Potato Virus X (PVX). Preferably, the viral vector is at least one of a Tobacco Rattle Virus (TRV), a Tobacco Mosaic Virus (TMV), a geminivirus, a Potato Virus X (PVX) and a Sonchus Yellow Net Virus (SYNV).

Preferably, the vector does not have a modification that compromises, or significantly compromises, its systemic mobility. Therefore, preferably the vector for use in the invention comprises a functional coat protein. Alternatively, the vector for use in the invention does not express a functional coat protein. The sequence encoding the coat protein may comprise a mutation to abolish the expression of a functional coat protein. The mutation may be at least one of a deletion, an addition, a substitution or a combination thereof, preferably resulting in an early stop codon. Preferably at least part of the sequence encoding the coat protein is deleted. Preferably, at least 2%, 5%, 10%, 20%, 30%, 40%, 50%, 60%, 70%, 80%, 90% or 100% of the sequence encoding the viral coat protein is deleted from the viral genome. Although deletion of the coat protein may compromise systemic mobility, it may be desired as may increase packaging capacity. Preferably, the viral vector comprising a mutation, preferably a deletion, of at least part of a sequence encoding the coat protein is a Tobamovirus virus or a Tobravirus virus, optinally a Tobacco Mosaic Virus (TMV), modified to comprise said mutation or deletion. A preferred viral vector is the TMV RNA-based overexpression vector (TRBO), e.g. as described in Lindbo (TRBO: A High-Efficiency Tobacco Mosaic Virus RNA-Based Overexpression Vector, Plant Physiol, 2007; 145(4):1232-40). Thus the viral vector for use in the method of the invention may comprise a deletion of one or more genes, e.g. to increase the packaging capacity of the virus, to improve genome stability and/or to increase gene expression.

Infection of a plant cell with a suitable vector preferably results in high level expression of at least one element of the site-directed nuclease, preferably at least one of a CRISPR-nuclease and a guide RNA in the plant cell. High level expression may for example be achieved by deleting the viral coat protein and/or the use of a strong promoter. The viral vector may be a self-replicating RNA as e.g. described in WO2018/226972, which is incorporated herein by reference.

### Step iii) Removing the Shoot Apical Meristem

After introducing the vector in step ii), the shoot apical meristem (or “growing point”) may be removed from the provided plant. The shoot apical meristem may be removed directly after introducing the vector in a cell of the plant, e.g. the time in between steps ii) and iii) may be less than about 24 h, 28 h, 12 h, 6 h or even less than about 3 h. Preferably, the plant produces (replicates) the introduced vector prior to removing the shoot apical meristem. Therefore there may be one or more days in between step ii) and step iii) of the method of the invention. Preferably, there are about 1, 2, 3, 4 or 5 weeks in between step ii) and step iii). Preferably, there are at least 1, 2, 3, 4, 5, 6, 7, 8, 9, 10, 11, 12, 13, 14, 15, 16, 17, 18, 19, 20 or 21 days in between step ii) and step iii). Preferably, there are about 1-21, 2-15 or about 7-10 days in between step ii) and step iii).

Step iii) of the method of the invention concerns the removal of the shoot apical meristem of the provided plant.

Removal of the shoot apical meristem may additionally result in the removal of one or more axillary meristems. Preferably, removal of the shoot apical meristem does not results in the removal of all axillary meristems of the provided plant. Preferably, removal of the shoot apical meristem preferably does not initiate regeneration of the provided plant. After removing the shoot apical meristem, the provided plant preferably still comprises one or more axillary meristems. Preferably, after removing the shoot apical meristem, the provided plant comprises the cotyledonous axillary meristems. Optionally, after removing the shoot apical meristem, the provided plant, apart from comprising cotyledonous axillary meristem, does not comprise any further axillary shoot meristem.

The skilled person is well aware of methods suitable for removing the shoot apical meristem of a plant. Preferably, the removal of the shoot apical meristem is by decapitation of the plant at a first location. Preferably, the first location is above the cotyledons. Preferably, the first location is just above the cotyledons. Preferably, the (first) location of decapitating the plant is in the epicotyl. Preferably, the first location is below the first true leaves, preferably above the cotyledons and below the first true leaves. Preferably, the (first) location for decapitating the plant is less than about 20 cm, 15 cm, 10 cm, 9 cm, 8 cm, 7 cm, 6 cm, 5 cm, 4 cm, 3 cm, 2 cm, 1 cm, 0.9 cm, 0.8 cm, 0.7 cm, 0.6 cm, 0.5 cm, 0.4 cm, 0.3 cm, 0.2 cm or less than about 0.1 cm above the cotyledons.

### Step iv) Shoot Regeneration

The step of removing the shoot apical meristem is followed by a step iv) of regenerating a shoot at a second location in the plant resulting from step iii). Shoot regeneration at the second location is preferably induced by wounding the plant at the second location. Preferably, said wounding is decapitation at the second location. Preferably, the shoot regeneration is a de novo formation of a shoot. Preferably in step iv), the plant is decapitated to induce shoot formation. Preferably the decapitation is at a second location, wherein preferably the second location is below the cotyledons. Preferably, step iv) comprises removal of a (shoot) axillary meristem, preferably all (shoot) axillary meristem, including potential axillary meristem that developed into dominant, new shoot apical meristem as a result of the removal of the shoot apical meristem in step iii), thereby inducing meristem formation from somatic cells, preferably a somatic cell comprising the genome modification. In particular, the shoot is preferably regenerated from a meristem formed from at least one somatic cell. Preferably said at least one somatic cell comprises a genetic modification induced by the site-directed nuclease.

Optionally, step iii) of removing the apical meristem is directly followed by step iv) of regenerating a shoot at a second location in a plant, e.g. step iii) and iv) may be performed on the same day, e.g. the time in between steps iii) and iv) may be less than about 24 h, 28, 12 h, 6 h or even less than about 3 h.

Preferably, the time between step iii) and iv) may be one or more days, such that the provided plant comprises one or more cells comprising a genetic modification induced by the site-directed nuclease at the second location.

Therefore there may be one or more days in between step iii) and step iv) of the method of the invention. Optionally, there are about 1, 2, 3, 4 or 5 weeks in between step iii) and step iv). Preferably, there are at least 1, 2, 3, 4, 5, 6, 7, 8, 9, 10, 11, 12, 13, 14, 15, 16, 17, 18, 19, 20, 21, 25, 30, 35, 40, 45 or 50 days in between step iii) and step iv). Preferably, there are about 1-50, 2-40, 3-30, 4-25, 5-20, 6-15, 7-14, 1-7 or 1-5 days in between step iii) and step iv).

Shoot regeneration is preferably induced at a second location in the provided plant (see also FIG. 1 for a schematic representation of an embodiment of the invention). The second location is thus a location that allows the regeneration of a shoot. Preferably, the location does not allow the regeneration of a root. The location is preferably in the shoot system of a plant. Preferably, the second location is not in the root system of a plant and/or is not the removal of a root apical meristem. Preferably, the first location and the second location are in close vicinity of each other. The first and second location can both be located in the epicotyl of the provided plant or can both be located in the hypocotyl. The first location may be in the epicotyl and the second location may be in the hypocotyl. Preferably, the first location is above the cotyledons, and the second location is below the cotyledons. Preferably, the first location is just above the cotyledons, and the second location is just below the cotyledons, wherein “just” is within about 3 cm, 2 cm, 1 cm, 0.5 cm, 0.4 cm, 0.3 cm, 0.2 cm or about 0.1 cm of the cotyledon. Preferably, the cotyledons are located in between the first and the second location of the plant provided in step i).

The distance between the first and second location is preferably less than about 20 cm, 15 cm, 10 cm, 9 cm, 8 cm, 7 cm, 6 cm, 5 cm, 4 cm, 3 cm, 2 cm, 1 cm, 0.9 cm, 0.8 cm, 0.7 cm, 0.6 cm, 0.5 cm, 0.4 cm, 0.3 cm, 0.2 cm or less than about 0.1 cm. Preferably, the distance between the first and the second location is about 0.1 cm-20 cm, 0.5 cm-10 cm, 1 cm-8 cm or about 2 cm-6 cm.

The skilled person is well aware of methods for regenerating a shoot in a plant. Such conventional methods include, but are not limited, wounding (preferably decapitating) the provided plant at the second location. Decapitation is understood herein as the removal of an axillary meristem, preferably the removal all (shoot) axillary meristems of the provided plant. Preferably, all axillary meristems are removed to prevent the development of a shoot from an existing meristem. Preferably there are no shoots formed from an pre-existing meristem. Preferably, there are one or more shoots formed from somatic cells that have been converted into new meristems. Preferably at least one or more of these somatic cells comprise a genetic modification induced by the site-directed nuclease, and hence preferably the meristems formed from said one or more somatic cells comprises the genetic modification.

Alternatively or in addition, the provided plant may be wounded in step iv) to stimulate shoot regeneration. Wounding is a well-known step in tissue-culture techniques and the skilled person knows how to wound a plant cell and to induce wound stress.

The second location is preferably in the hypocotyl of the plant provided in step i). Preferably, the second location is a location below the cotyledons. Preferably, the second location for decapitating the plant is less than about 20 cm, 15 cm, 10 cm, 9 cm, 8 cm, 7 cm, 6 cm, 5 cm, 4 cm, 3 cm, 2 cm, 1 cm, 0.9 cm, 0.8 cm, 0.7 cm, 0.6 cm, 0.5 cm, 0.4 cm, 0.3 cm, 0.2 cm or less than about 0.1 cm below the cotyledons.

At least step iv) is preferably performed under conditions that allow for shoot regeneration. These conditions are understood herein as at least being the minimal requirements of the provided plant to regenerate, which in general at least include normal growth conditions of said plant.

The regeneration process can occur directly from parental tissue or indirectly, e.g. via the formation of callus. The shoot regeneration in step iv) of the method of the invention thus may involve a callus stage. The shoot may thus form from the callus. This formation of callus, e.g. after wounding (preferably decapitation), may occur spontaneously, i.e. in the absence of one or more externally supplied plant hormones. Similarly the formation of shoots, e.g. after callus formation, may occur spontaneously, thus in the absence of one or more externally supplied plant hormones.

Alternatively, the formation of callus in the method of the invention may be induced and/or augmented in the presence of one or more plant hormones. Alternatively or in addition, the formation of shoots may be induced and/or augmented in the presence of one or more plant hormones. Preferably, plant hormones for shoot regeneration used in step iv) may be one or more cytokinins or one or more auxins, or a combination of one or more cytokinins and one or more auxins.

The one or more cytokinins that may be used in the method of the invention can be at least one of an adenine-type cytokinin and a phenylurea-type cytokinin. Similarly, the cytokinin can be a naturally produced phytohormone or can be a synthesized compound. The adenine-type cytokinin can be a phytohormone that is synthesized in at least one of roots, seeds and fruits. In addition, cambium and other actively dividing tissues can also synthesize cytokinins. A non-limiting example of a naturally occurring adenine-type cytokinin is Zeatin as well as its metabolic precursor 2iP. Non-limiting examples of synthetic adenine-type cytokinins are kinetin and 6-benzylaminopurine (BAP). Substituted urea compounds, such as thidiazuron and CPPU do not occur in plants but can act as cytokinins in tissue culture. The adenine-type cytokinin can be selected from the group consisting of kinetin, zeatin, trans-zeatin, cis-zeatin, dihydrozeatin, 6-benzylaminopurine and 2iP, and combinations thereof. The phenylurea-type cytokinin can be diphenylurea or thidiazuron. It is known in the art that the type of added cytokinin is dependent on the type of plant cell and the skilled person can straightforwardly select the suitable cytokinin(s), if needed.

The one or more auxins that may be used in the method of the invention can be an endogenously synthesized auxin. The endogenously synthesized auxin can be selected from the group consisting of indole-3-acetic acid (IAA), 4-chloroindole-3-acetic acid, phenylacetic acid, indole-3-butyric acid and indole-3-propionic acid. The auxin can be a synthetic auxin, e.g. an auxin analog. The synthetic auxin can be at least one of 1-naphthaleneacetic acid, 2,4-dichlorophenoxyacetic acid (2,4-D), α-Naphthalene acetic acid (α-NAA), 2-Methoxy-3,6-dichlorobenzoic acid (dicamba), 4-Amino-3,5,6-trichloropicolinic acid (tordon or picloram), 1-naphthaleneacetic acid (NAA), indole-3-butyric acid (IBA) and 2,4,5-trichlorophenoxyacetic acid (2,4,5-T). The auxin can be 1-naphthaleneacetic acid (NAA).

For shoot organogenesis by a combination of cytokinin and auxin, the cytokinin to auxin ratio preferably is >1 (Dodds, J H and Roberts, L W (1985) Experiments in plant tissue culture. Cambridge University Press, Cambridge, UK).

Step iv) may be performed using conditions allowing for callus formation at the second location of the provided plant. Optionally, step iv) is performed using minimal conditions allowing for callus formation at the second location. Optionally, step iv) is performed using optimal conditions for callus formation at the second location of the provided plant.

Step iv) may be performed using conditions allowing for shoot formation at the second location of the provided plant. Preferably, step iv) is performed using minimal conditions allowing for shoot formation at the second location. In a preferred embodiment, step iv) of the method of the invention is performed using optimal conditions for shoot formation at the second location of the provided plant.

Preferably, at least one of the shoots regenerated from the plant comprises one or more cells having a genetic modification induced by the site-directed nuclease. Optionally, at least one of the shoots regenerated from the plant comprises multiple cells having a genetic modification induced by the site-directed nuclease, wherein the resulting modifications (optionally being a nucleotide substitution, insertion and/or deletion, e.g. an indel) may differ between the multiple cells. Different modifications induced by the single site-directed nuclease of the method of the invention may occur in case a DSB induced by said site-directed nuclease is repaired in a process indicated as NHEJ (non-homologous end joining) in different ways in different cells, optionally resulting in various nucleotide substitutions, deletions and/or insertions.

Preferably, a shoot regenerated in step iv) of the method of the invention comprises more genetically modified cells as compared to a regenerated shoot of a control plant. Preferably, the shoot of the control plant is regenerated using the same method as described herein, with the exception that step iii) of the method is omitted. There is preferably a difference of at least about 2%, 5%, 10%, 20%, 30%, 40%, 50%, 60%, 70%, 80%, 90%, 95% or 100% between the percentage of genetically modified cells in the shoot produced by the method of the invention, as compared to the percentage of genetically modified cells of the shoot produced by the control plant. As a non-limiting example, 50% of the cells of the shoot regenerated by the method of the invention can have a genetic modification induced by the site-directed nuclease. Using the same experimental setting, with the exception that step iii) is omitted (indicated herein as control method), 30% of the cells may have a genetic modification induced by the site-directed nuclease. In this non-limiting example, there is thus a difference of 20% in number of cells from the regenerated shoots that have a genetic modification between the method of the invention (double decapitation) and the control method (single decapitation). Preferably, the shoot of a control plant is a shoot regenerated from a, preferably otherwise identical, plant subjected to the same method as defined herein, with the exception that step iii) is omitted. Optionally, if a plant regenerates more than one shoot, the percentage of genetically modified cells in a shoot of a plant as defined herein may be understood herein as the average of all shoots of said plant. Preferably, at least about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95% or 100% of the cells of a shoot formed at the second location have a genetic modification induced by the site-directed nuclease. Preferably, all cells of the formed shoot comprise a genetic modification induced by the site-directed nuclease.

Preferably, at least one of the shoots regenerated from the plant comprises one or more cells having a genetic modification induced by the site-directed nuclease. Preferably, at least about 2%, 5%, 10%, 20%, 30%, 40%, 50%, 60%, 70%, 80%, 90%, 95% or 100% of the regenerated shoots comprise one or more cells having a genetic modification induced by the site-directed nuclease. Preferably, all regenerated shoots comprise one or more cells having a genetic modification induced by the site-directed nuclease. The method of the invention preferably increases the number of shoots having one or more genetically modified cells. Therefore preferably the method of the invention results in an increased number of regenerated shoots that have one or more genetically modified cells as compared to the same method, with the exception that step iii) is omitted. There is preferably a difference of at least about 2%, 5%, 10%, 20%, 30%, 40%, 50%, 60%, 70%, 80%, 90%, 95% or 100% between the percentage shoots having one or more genetically modified cells produced by the method of the invention, as compared to the percentage of shoots having one or more genetically modified cells produced by the control plant. Preferably, the shoots of a control plant are the regenerated shoots from a, preferably otherwise identical, plant subjected to the same method as defined herein, with the exception that step iii) is omitted.

In case more than one plant is provided in step i), the percentage of modified cells in a shoot may be understood herein as the average percentage of modified cells in a shoot, as determined after analysis of all regenerated shoots of all plants. Equally, the percentage of shoots comprising one or more genetically modified cells may be understood herein as the average percentage of shoots comprising one or more genetically modified cells, after analysis of all regenerated shoots of all plants.

The method of the invention may further comprise a step of selecting the shoot comprising one or more cells having a genetic modification induced by the site-directed nuclease, optionally selecting a shoot comprising one or more cells having a particular desired genetic modification, for instance a particular indel or single nucleotide polymorphism of interest. Preferably, the method of the invention comprises a step of identifying and/or selecting the shoot comprising one or more meristem cells having the genetic modification. Optionally said shoot may be isolated. Preferably, the selected shoot is regenerated into a plant. Preferably, the selected shoot is regenerated into a plant that gives rise to gametes bearing or having the genetic modification. Preferably, the selected shoot is grown as part of the plant of the method of the invention where the shoot originates from, to develop an inflorescence bearing at least one gamete having the genetic modification. Said gamete may be a male gamete in a pollen grain or a female gamete in the ovule of a flower. Optionally, said pollen or ovule is part of a cross giving rise to a seed and/or progeny comprising the genetic modification.

The step of selecting the shoot can be performed using any conventional method known to the skilled person. The selection may comprise a step of determining a phenotypic and/or a molecular characteristic of the shoot. The selection may comprise a step of detecting the genetic modification. The selection may comprise a step of genotyping. Detection of the genetic modification and/or genotyping may be performed using any conventional method known to the skilled person, such as, but not limited to, using conventional (deep) sequencing methods.

Alternatively or in addition, the method of the invention as defined herein may comprise a step of growing the plant after step iv). Preferably after regenerating a shoot at the second location in the provided plant, optionally after decapitating the provided plant at the second location, the plant may be cultured or “grown” for at least about 1, 2, 3, 4, 5, 6, 7, 8, 9, or about 10 weeks, preferably for at least about 1, 2, 3, 4, 5, 6, 7, 8, 9 or about 10 months. Preferably after regenerating a shoot at the second location in the provided plant, optionally after decapitating the provided plant at the second location, the plant may be grown towards at least one of a vegetative plant, a budding plant, a flowering plant or a ripening plant. The plant may be grown to produce pollen and/or eggs. Preferably the method comprises a step of growing the plant to produce seed, optionally via a step of and (self-)fertilizing an egg cell comprising the genetic modification. Preferably the step of seed production does not involve an essential or exclusively biological process.

The produced seed preferably comprises one or more cells having the genetic modification induced by the site-specific nuclease of the method of the invention. Preferably, the cells of the embryo comprise said genetic modification. Optionally, the integument may comprise said genetic modification. Preferably all cells of the produced seed comprise the genetic modification induced by the site-specific nuclease of the method of the invention.

The method may further comprise a step of growing a progeny plant from the produced seed, wherein the progeny plant comprises one or more cells having the genetic modification induced by the site-specific nuclease of the invention. Preferably, the progeny plant is not, or is not exclusively, obtainable by an essential biological process.

In an aspect, the invention pertains to a plant obtainable by the method of the invention, i.e. a plant comprising one or more regenerated shoots having a genetic modification induced by the site-directed nuclease. The plant of the invention preferably differs at least from a plant occurring in nature, in that it contains at least one mutation in a sequence of interest. Preferably, the plant of the invention is not, or is not exclusively, obtained by an essentially biological process.

In a further aspect, the invention relates to a shoot obtainable by the method of the invention, wherein the shoot comprises one or more cells comprising a genetic modification induced by the site-directed nuclease of the method of the invention. Optionally, the shoot may be grown into a plant comprising the genetic modification.

The invention further pertains to a seed or a progeny plant produced by the method of the invention comprising the genetic modification. Preferably, the seed and the progeny plant are not, or are not exclusively, obtainable by an essential biological process.

The present invention has been described above with reference to a number of exemplary embodiments. Modifications and alternative implementations of some parts or elements are possible, and are included in the scope of protection as defined in the appended claims.

### FIGURE LEGENDS

FIG. 1 Schematic representation of an embodiment of the invention.

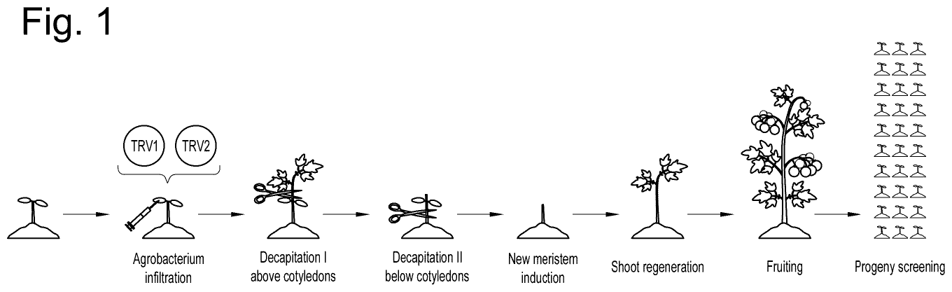

FIG. 2 Shoots containing edits in at least one of the target genes for the double decapitation approach (left, 15 shoots) and the single decapitation approach (right, 4 shoots). The y axis represents the frequency of edits in each target gone (Lin5, ETR1 and RR-A).

### EXAMPLES

### Experimental Procedure

Seeds of tomato line Moneyberg TMV+ (The Netherlands) expressing Cas9 (SEQ ID NO: 4) under the control of a Parsley ubiquitin promoter (Fauser et al. Plant J. 2014; 79:348-359) were sown in small pots and placed in a climate-controlled compartment for 7-10 days for germination.

Agrobacterium strains containing the plasmid pTRV1 (accession AF406991.1) or pTRV2 (accession number: AF406991) were grown overnight at 28° C., 225 rpm in LB medium supplemented with kanamycin (Liu et al. Plant J. 2002 May; 30 (4): 415-429). pTRV1 and pTRV2 encode two RNA molecules that together form the genome of the bi-partite virus TRV. pTRV1 encodes for TRV RNA1. The RNA molecule “RNA1” comprises the elements known in the art to be essential for replication and movement of the virus. pTRV2 encodes for TRV RNA2. The RNA molecule “RNA2” expresses single guide (sg) RNAs targeting respectively the LIN5, ETR1 and RRA gene, having the seed sequence of CCTGACGATGAAATTAAGAA (SEQ ID NO: 12), ACGAAGTATATCAACTCCAC (SEQ ID NO: 13) and TATTGTATGCCTGGGATGAC (SEQ ID NO: 14), respectively, under the control of the PEBV sub-genomic promoter (Ali et al. Molecular Plant, 2015; 8 (8): p1288-1291).

The next day bacteria were harvested by centrifugation at 3400 rcf for 5 minutes at room temperature and the bacterial pellets were re-suspended in 10 mL of infiltration buffer (10 mM MES, 10 mM MgCl₂, pH 5.6 supplemented with 200 μM acetosyringone).

TRV1 and TRV2 cultures were diluted to a final OD₆₀₀ of 0.8-0.9 using infiltration buffer and mixed 1:1. The mixture was incubated in infiltration buffer for 3-4 hours at room temperature prior to infiltration into the cotyledons of each plant.

After 10 days, 20 seedlings were decapitated above the cotyledons (for the double decapitation samples), and 20 seedlings were decapitated just below the cotyledons (for the single decapitation samples) using a single edge razor blade. Water was added to the tray which was then returned to the climate-controlled cabinet.

After an additional 3 days, the double-decapitation seedlings were decapitated just under the cotyledons using a single edge razor blade. The covered tray was returned to the climate-controlled cabinet. Both the single-and double-decapitation seedlings, once shoots started to form, were transferred to the greenhouse. Each shoot was individually labelled.

At the fruiting stage (or late flowering stage), leaf material, close to the young fruits or inflorescences, was collected. After DNA extraction, amplicons spanning the sgRNA cutting site for all 3 target genes were generated by PCR. After addition of sample tags and sequencing adapters by PCR, samples were pooled and sequenced on the Illumina MiSeq platform.

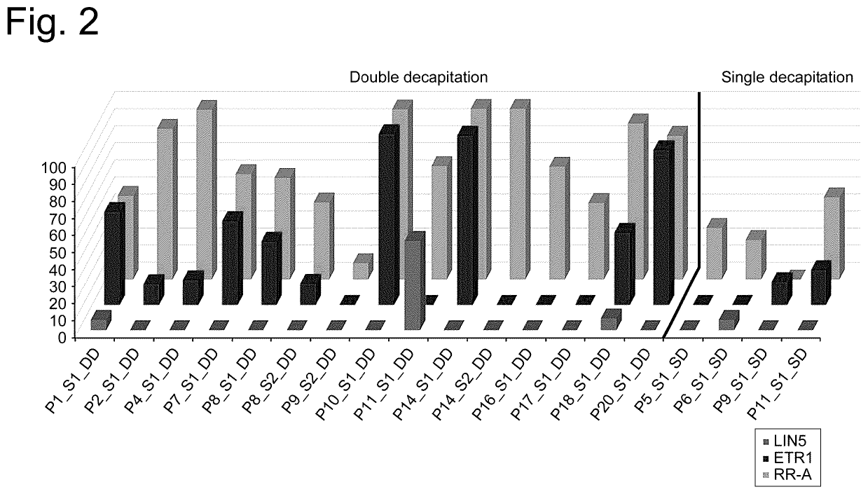

The frequency of the edits was determined as the percentage of reads with InDels at the expected cut site per amplicon. The results are shown in Table 1 and FIG. 2.

TABLE 1
Percentage of cells of the shoot comprising
the edit (% of genotype assay reads)

| Double-decapitation | | | | Single-decapitation | | | |
|---|---|---|---|---|---|---|---|
| | Gene | | | | Gene | | |
| Shoot | LIN5 | ETR1 | RR-A | Shoot | LIN5 | ETR1 | RR-A |
| P1-S1 | 6 | 54.4 | 49.1 | P5-S1 | — | — | 30.3 |
| P2-S1 | — | 12.1 | 88.4 | P6-S1 | 6 | — | 23.2 |
| P7-S1 | — | 49 | 61.8 | P9-S1 | — | 13.3 | — |
| P8-S1 | — | 37 | 59.6 | P11-S1 | — | 20.3 | 48.2 |
| P8-S2 | — | 12.3 | 45.2 | | | | |
| P9-S2 | — | — | 9.6 | | | | |
| P10-S1 | — | 100 | 99.7 | | | | |
| P11-S1 | 52.3 | — | 66.5 | | | | |
| P14-S1 | — | 99.1 | 100 | | | | |
| P14-S2 | — | — | 100 | | | | |
| P16-S1 | — | — | 66.2 | | | | |
| P17-S1 | — | — | 44.9 | | | | |
| P18-S1 | 6.9 | 42.2 | 91.5 | | | | |
| P20-S1 | — | 90.6 | 84.2 | | | | |
| P24-S1 | — | 14.4 | 99.5 | | | | |

In addition, the number edited shoots was determined as indicated in Table 2 below.

TABLE 2
Number of shoots comprising edits

| | Single | Double |
|---|---|---|
| | decapitation | decapitation |
| Regenerated shoots | 10 | 24 |
| Regenerated shoots with edits | 4 | 15 |
| Percentage of regenerated | 40% | 63% |
| shoots with edits | | |

As shown in Table 1 and FIG. 2, many shoots show edits at frequencies above 40% in the double decapitation (75%), while only 16% of the edits exceed 40% in the single decapitation approach. The method of the invention thus clearly increases the number of edits per regenerated shoot.

Strikingly, also the percentage regenerated roots comprising edits increased significantly using the method of the invention (Table 2 shows an increase of 23%). The method of the invention thus results in an increase in the number of edits per shoot as well as the number of shoots comprising edited cells.

1. A method for producing a shoot of a plant, wherein the shoot comprises one or more cells having a genetic modification, and wherein the method comprises:
(i) introducing in a cell of a plant a vector expressing at least one of a guide element and nuclease element of a site-directed nuclease;
(ii) removing the shoot apical meristem of the plant at a first location; and
(iii) regenerating a shoot at a second location in the plant at the second location, wherein the shoot comprises one or more cells having a genetic modification.

2. The method according to claim 1, wherein removal of the shoot apical meristem is by decapitation.

3. The method according to claim 1, wherein regeneration of the shoot is by wounding.

4. The method according to claim 1, wherein regeneration of the shoot is by decapitation.

5. The method according to claim 1, wherein the first location is above the cotyledons.

6. The method according to claim 5, wherein the first location is in the epicotyl above the cotyledons and below the first true leaves.

7. The method according to claim 1, wherein the second location is below the cotyledons.

8. The method according to claim 7, wherein the second location is in the hypocotyl below the cotyledons.

9. The method according to claim 1, wherein the site-directed nuclease is selected from the group consisting of a CRISPR-nuclease complex, an Argonaute, a Zinc finger nuclease, a TALEN and a meganuclease.

10. The method according to claim 1, wherein the site-specific nuclease is a CRISPR-nuclease complex, wherein the plant expresses the nuclease element of the complex, and the vector expresses a gRNA.

11. The method according to claim 1, wherein the period between the introducing (i) and the removing (ii) is 1-21 days.

12. The method according to claim 11, wherein the period is 1-7 days.

13. The method according to claim 1, wherein the plant is a seedling.

14. The method according to claim 1, wherein the introduced vector is a virus.

15. The method according to claim 14, wherein the virus is selected from the group consisting of a Tobacco Rattle Virus (TRV), a Tobacco Mosaic Virus (TMV), a Potato virus X (PVX), a geminivirus, a Sonchus yellow net virus (SYNV).

16. The method according to claim 15, wherein the virus is a tobacco mosaic virus RNA-based overexpression vector (TRBO).

17. The method according to claim 1, wherein the introducing is by contacting the cotyledon with an agrobacterium or virus particle carrying the vector.

18. The method according to claim 1, further comprising (iv) selecting the shoot comprising one or more cells having a genetic modification and regenerating a plant thereof.

19. The method according to claim 1, further comprising (iv) growing the plant to produce a gamete comprising the genetic modification.

20. The method according to claim 19, further comprising (v) growing a seed from the gamete and optionally a progeny plant from the seed, wherein the seed and optional progeny plant comprises one or more cells having the genetic modification.

21. A plant having a shoot comprising one or more cells having a genetic modification, wherein the plant is obtainable by the method according to claim 1.


SEQUENCE LISTING (14 sequences)

SEQ ID NO: 1 - protein, 14 aa, synthetic construct
  REGION 1..14: note = SV40 Large T-antigen
MEDPTMAPKKKRKV

SEQ ID NO: 2 - protein, 7 aa, synthetic construct
  REGION 1..7: note = monopartite NLS
PKKKRKV

SEQ ID NO: 3 - protein, 16 aa, synthetic construct
  REGION 1..16: note = nucleoplasmin NLS
KRPAATKKAGQAKKKK

SEQ ID NO: 4 - protein, 1368 aa, synthetic construct [sequence omitted]

SEQ ID NO: 5 - DNA, 4104 bp, synthetic construct
  misc_feature 1..4104: note = Cas9
  source 1..4104: mol_type = other DNA
atggataaaaaatatagcattggtctggatattggtaccaatagcgttggttgggcagttattaccgatgaatataaagttccgagcaaaaaatttaaagttctgggtaataccgatcgtcatagcattaaaaaaaatctgattggtgcactgctgtttgatagcggtgaaaccgcagaagcaacccgtctgaaacgtaccgcacgtcgtcgttatacccgtcgtaaaaatcgtatttgttatctgcaggaaatttttagcaatgaaatggcaaaagttgatgatagcttttttcatcgtctggaagaaagctttctggttgaagaagataaaaaacatgaacgtcatccgatttttggtaatattgttgatgaagttgcatatcatgaaaaatatccgaccatttatcatctgcgtaaaaaactggttgatagcaccgataaagcagatctgcgtctgatttatctggcactggcacatatgattaaatttcgtggtcattttctgattgaaggtgatctgaatccggataatagcgatgttgataaactgtttattcagctggttcagacctataatcagctgtttgaagaaaatccgattaatgcaagcggtgttgatgcaaaagcaattctgagcgcacgtctgagcaaaagccgtcgtctggaaaatctgattgcacagctgccgggtgaaaaaaaaaatggtctgtttggtaatctgattgcactgagcctgggtctgaccccgaattttaaaagcaattttgatctggcagaagatgcaaaactgcagctgagcaaagatacctatgatgatgatctggataatctgctggcacagattggtgatcagtatgcagatctgtttctggcagcaaaaaatctgagcgatgcaattctgctgagcgatattctgcgtgttaataccgaaattaccaaagcaccgctgagcgcaagcatgattaaacgttatgatgaacatcatcaggatctgaccctgctgaaagcactggttcgtcagcagctgccggaaaaatataaagaaattttttttgatcagagcaaaaatggttatgcaggttatattgatggtggtgcaagccaggaagaattttataaatttattaaaccgattctggaaaaaatggatggtaccgaagaactgctggttaaactgaatcgtgaagatctgctgcgtaaacagcgtacctttgataatggtagcattccgcatcagattcatctgggtgaactgcatgcaattctgcgtcgtcaggaagatttttatccgtttctgaaagataatcgtgaaaaaattgaaaaaattctgacctttcgtattccgtattatgttggtccgctggcacgtggtaatagccgttttgcatggatgacccgtaaaagcgaagaaaccattaccccgtggaattttgaagaagttgttgataaaggtgcaagcgcacagagctttattgaacgtatgaccaattttgataaaaatctgccgaatgaaaaagttctgccgaaacatagcctgctgtatgaatattttaccgtttataatgaactgaccaaagttaaatatgttaccgaaggtatgcgtaaaccggcatttctgagcggtgaacagaaaaaagcaattgttgatctgctgtttaaaaccaatcgtaaagttaccgttaaacagctgaaagaagattattttaaaaaaattgaatgttttgatagcgttgaaattagcggtgttgaagatcgttttaatgcaagcctgggtacctatcatgatctgctgaaaattattaaagataaagattttctggataatgaagaaaatgaagatattctggaagatattgttctgaccctgaccctgtttgaagatcgtgaaatgattgaagaacgtctgaaaacctatgcacatctgtttgatgataaagttatgaaacagctgaaacgtcgtcgttataccggttggggtcgtctgagccgtaaactgattaatggtattcgtgataaacagagcggtaaaaccattctggattttctgaaaagcgatggttttgcaaatcgtaattttatgcagctgattcatgatgatagcctgacctttaaagaagatattcagaaagcacaggttagcggtcagggtgatagcctgcatgaacatattgcaaatctggcaggtagcccggcaattaaaaaaggtattctgcagaccgttaaagttgttgatgaactggttaaagttatgggtcgtcataaaccggaaaatattgttattgaaatggcacgtgaaaatcagaccacccagaaaggtcagaaaaatagccgtgaacgtatgaaacgtattgaagaaggtattaaagaactgggtagccagattctgaaagaacatccggttgaaaatacccagctgcagaatgaaaaactgtatctgtattatctgcagaatggtcgtgatatgtatgttgatcaggaactggatattaatcgtctgagcgattatgatgttgatcatattgttccgcagagctttctgaaagatgatagcattgataataaagttctgacccgtagcgataaaaatcgtggtaaaagcgataatgttccgagcgaagaagttgttaaaaaaatgaaaaattattggcgtcagctgctgaatgcaaaactgattacccagcgtaaatttgataatctgaccaaagcagaacgtggtggtctgagcgaactggataaagcaggttttattaaacgtcagctggttgaaacccgtcagattaccaaacatgttgcacagattctggatagccgtatgaataccaaatatgatgaaaatgataaactgattcgtgaagttaaagttattaccctgaaaagcaaactggttagcgattttcgtaaagattttcagttttataaagttcgtgaaattaataattatcatcatgcacatgatgcatatctgaatgcagttgttggtaccgcactgattaaaaaatatccgaaactggaaagcgaatttgtttatggtgattataaagtttatgatgttcgtaaaatgattgcaaaaagcgaacaggaaattggtaaagcaaccgcaaaatattttttttatagcaatattatgaatttttttaaaaccgaaattaccctggcaaatggtgaaattcgtaaacgtccgctgattgaaaccaatggtgaaaccggtgaaattgtttgggataaaggtcgtgattttgcaaccgttcgtaaagttctgagcatgccgcaggttaatattgttaaaaaaaccgaagttcagaccggtggttttagcaaagaaagcattctgccgaaacgtaatagcgataaactgattgcacgtaaaaaagattgggatccgaaaaaatatggtggttttgatagcccgaccgttgcatatagcgttctggttgttgcaaaagttgaaaaaggtaaaagcaaaaaactgaaaagcgttaaagaactgctgggtattaccattatggaacgtagcagctttgaaaaaaatccgattgattttctggaagcaaaaggttataaagaagttaaaaaagatctgattattaaactgccgaaatatagcctgtttgaactggaaaatggtcgtaaacgtatgctggcaagcgcaggtgaactgcagaaaggtaatgaactggcactgccgagcaaatatgttaattttctgtatctggcaagccattatgaaaaactgaaaggtagcccggaagataatgaacagaaacagctgtttgttgaacagcataaacattatctggatgaaattattgaacagattagcgaatttagcaaacgtgttattctggcagatgcaaatctggataaagttctgagcgcatataataaacatcgtgataaaccgattcgtgaacaggcagaaaatattattcatctgtttaccctgaccaatctgggtgcaccggcagcatttaaatattttgataccaccattgatcgtaaacgttataccagcaccaaagaagttctggatgcaaccctgattcatcagagcattaccggtctgtatgaaacccgtattgatctgagccagctgggtggtgat

SEQ ID NO: 6 - protein, 1082 aa, synthetic construct
  REGION 1..1082: note = Cas9
MKYKIGLDIGITSIGWAVINLDIPRIEDLGVRIFDRAENPKTGESLALPRRLARSARRRLRRRKHRLERIRRLFVREGILTKEELNKLFEKKHEIDVWQLRVEALDRKLNNDELARILLHLAKRRGFRSNRKSERTNKENSTMLKHIEENQSILSSYRTVAEMVVKDPKFSLHKRNKEDNYTNTVARDDLEREIKLIFAKQREYGNIVCTEAFEHEYISIWASQRPFASKDDIEKKVGFCTFEPKEKRAPKATYTFQSFTVWEHINKLRLVSPGGIRALTDDERRLIYKQAFHKNKITFHDVRTLLNLPDDTRFKGLLYDRNTTLKENEKVRFLELGAYHKIRKAIDSVYGKGAAKSFRPIDFDTFGYALTMFKDDTDIRSYLRNEYEQNGKRMENLADKVYDEELIEELLNLSFSKFGHLSLKALRNILPYMEQGEVYSTACERAGYTFTGPKKKQKTVLLPNIPPIANPVVMRALTQARKVVNAIIKKYGSPVSIHIELARELSQSFDERRKMQKEQEGNRKKNETAIRQLVEYGLTLNPTGLDIVKFKLWSEQNGKCAYSLQPIEIERLLEPGYTEVDHVIPYSRSLDDSYTNKVLVLTKENREKGNRTPAEYLGLGSERWQQFETFVLTNKQFSKKKRDRLLRLHYDENEENEFKNRNLNDTRYISRFLANFIREHLKFADSDDKQKVYTVNGRITAHLRSRWNFNKNREESNLHHAVDAAIVACTTPSDIARVTAFYQRREQNKELSKKTDPQFPQPWPHFADELQARLSKNPKESIKALNLGNYDNEKLESLQPVFVSRMPKRSITGAAHQETLRRYIGIDERSGKIQTVVKKKLSEIQLDKTGHFPMYGKESDPRTYEAIRQRLLEHNNDPKKAFQEPLYKPKKNGELGPIIRTIKIIDTTNQVIPLNDGKTVAYNSNIVRVDVFEKDGKYYCVPIYTIDMMKGILPNKAIEPNKPYSEWKEMTEDYTFRFSLYPNDLIRIEFPREKTIKTAVGEEIKIKDLFAYYQTIDSSNGGLSLVSHDNNFSLRSIGSRTLKRFEKYQVDVLGNIYKVRGEKRVGVASSSHSKAGETIRPL

SEQ ID NO: 7 - protein, 1300 aa, synthetic construct
  REGION 1..1300: note = Cpf1
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 8 - DNA, 3900 bp, synthetic construct
  misc_feature 1..3900: note = Cpf1
  source 1..3900: mol_type = other DNA
atgagcatttatcaggaatttgttaataaatatagcctgagcaaaaccctgcgttttgaactgattccgcagggtaaaaccctggaaaatattaaagcacgtggtctgattctggatgatgaaaaacgtgcaaaagattataaaaaagcaaaacagattattgataaatatcatcagttttttattgaagaaattctgagcagcgtttgtattagcgaagatctgctgcagaattatagcgatgtttattttaaactgaaaaaaagcgatgatgataatctgcagaaagattttaaaagcgcaaaagataccattaaaaaacagattagcgaatatattaaagatagcgaaaaatttaaaaatctgtttaatcagaatctgattgatgcaaaaaaaggtcaggaaagcgatctgattctgtggctgaaacagagcaaagataatggtattgaactgtttaaagcaaatagcgatattaccgatattgatgaagcactggaaattattaaaagctttaaaggttggaccacctattttaaaggttttcatgaaaatcgtaaaaatgtttatagcagcaatgatattccgaccagcattatttatcgtattgttgatgataatctgccgaaatttctggaaaataaagcaaaatatgaaagcctgaaagataaagcaccggaagcaattaattatgaacagattaaaaaagatctggcagaagaactgacctttgatattgattataaaaccagcgaagttaatcagcgtgtttttagcctggatgaagtttttgaaattgcaaattttaataattatctgaatcagagcggtattaccaaatttaataccattattggtggtaaatttgttaatggtgaaaataccaaacgtaaaggtattaatgaatatattaatctgtatagccagcagattaatgataaaaccctgaaaaaatataaaatgagcgttctgtttaaacagattctgagcgataccgaaagcaaaagctttgttattgataaactggaagatgatagcgatgttgttaccaccatgcagagcttttatgaacagattgcagcatttaaaaccgttgaagaaaaaagcattaaagaaaccctgagcctgctgtttgatgatctgaaagcacagaaactggatctgagcaaaatttattttaaaaatgataaaagcctgaccgatctgagccagcaggtttttgatgattatagcgttattggtaccgcagttctggaatatattacccagcagattgcaccgaaaaatctggataatccgagcaaaaaagaacaggaactgattgcaaaaaaaaccgaaaaagcaaaatatctgagcctggaaaccattaaactggcactggaagaatttaataaacatcgtgatattgataaacagtgtcgttttgaagaaattctggcaaattttgcagcaattccgatgatttttgatgaaattgcacagaataaagataatctggcacagattagcattaaatatcagaatcagggtaaaaaagatctgctgcaggcaagcgcagaagatgatgttaaagcaattaaagatctgctggatcagaccaataatctgctgcataaactgaaaatttttcatattagccagagcgaagataaagcaaatattctggataaagatgaacatttttatctggtttttgaagaatgttattttgaactggcaaatattgttccgctgtataataaaattcgtaattatattacccagaaaccgtatagcgatgaaaaatttaaactgaattttgaaaatagcaccctggcaaatggttgggataaaaataaagaaccggataataccgcaattctgtttattaaagatgataaatattatctgggtgttatgaataaaaaaaataataaaatttttgatgataaagcaattaaagaaaataaaggtgaaggttataaaaaaattgtttataaactgctgccgggtgcaaataaaatgctgccgaaagttttttttagcgcaaaaagcattaaattttataatccgagcgaagatattctgcgtattcgtaatcatagcacccataccaaaaatggtagcccgcagaaaggttatgaaaaatttgaatttaatattgaagattgtcgtaaatttattgatttttataaacagagcattagcaaacatccggaatggaaagattttggttttcgttttagcgatacccagcgttataatagcattgatgaattttatcgtgaagttgaaaatcagggttataaactgacctttgaaaatattagcgaaagctatattgatagcgttgttaatcagggtaaactgtatctgtttcagatttataataaagattttagcgcatatagcaaaggtcgtccgaatctgcataccctgtattggaaagcactgtttgatgaacgtaatctgcaggatgttgtttataaactgaatggtgaagcagaactgttttatcgtaaacagagcattccgaaaaaaattacccatccggcaaaagaagcaattgcaaataaaaataaagataatccgaaaaaagaaagcgtttttgaatatgatctgattaaagataaacgttttaccgaagataaatttttttttcattgtccgattaccattaattttaaaagcagcggtgcaaataaatttaatgatgaaattaatctgctgctgaaagaaaaagcaaatgatgttcatattctgagcattgatcgtggtgaacgtcatctggcatattataccctggttgatggtaaaggtaatattattaaacaggatacctttaatattattggtaatgatcgtatgaaaaccaattatcatgataaactggcagcaattgaaaaagatcgtgatagcgcacgtaaagattggaaaaaaattaataatattaaagaaatgaaagaaggttatctgagccaggttgttcatgaaattgcaaaactggttattgaatataatgcaattgttgtttttgaagatctgaattttggttttaaacgtggtcgttttaaagttgaaaaacaggtttatcagaaactggaaaaaatgctgattgaaaaactgaattatctggtttttaaagataatgaatttgataaaaccggtggtgttctgcgtgcatatcagctgaccgcaccgtttgaaacctttaaaaaaatgggtaaacagaccggtattatttattatgttccggcaggttttaccagcaaaatttgtccggttaccggttttgttaatcagctgtatccgaaatatgaaagcgttagcaaaagccaggaattttttagcaaatttgataaaatttgttataatctggataaaggttattttgaatttagctttgattataaaaattttggtgataaagcagcaaaaggtaaatggaccattgcaagctttggtagccgtctgattaattttcgtaatagcgataaaaatcataattgggatacccgtgaagtttatccgaccaaagaactggaaaaactgctgaaagattatagcattgaatatggtcatggtgaatgtattaaagcagcaatttgtggtgaaagcgataaaaaattttttgcaaaactgaccagcgttctgaataccattctgcagatgcgtaatagcaaaaccggtaccgaactggattatctgattagcccggttgcagatgttaatggtaatttttttgatagccgtcaggcaccgaaaaatatgccgcaggatgcagatgcaaatggtgcatatcatattggtctgaaaggtctgatgctgctgggtcgtattaaaaataatcaggaaggtaaaaaactgaatctggttattaaaaatgaagaatattttgaatttgttcagaatcgtaataat

SEQ ID NO: 9 - protein, 1263 aa, synthetic construct
  REGION 1..1263: note = Mad7
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 10 - protein, 1274 aa, synthetic construct
  REGION 1..1274: note = Mad7
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYLSGGSPKKKRKV

SEQ ID NO: 11 - RNA, 23 nt, synthetic construct
  misc_feature 1..23: note = cleavable spacer
  source 1..23: mol_type = other RNA
tcgcggccgggtacgtgttgagc

SEQ ID NO: 12 - DNA, 20 bp, synthetic construct
  misc_feature 1..20: note = seed sequence LIN5
  source 1..20: mol_type = other DNA
cctgacgatgaaattaagaa

SEQ ID NO: 13 - DNA, 20 bp, synthetic construct
  misc_feature 1..20: note = seed sequence ETR1
  source 1..20: mol_type = other DNA
acgaagtatatcaactccac

SEQ ID NO: 14 - DNA, 20 bp, synthetic construct
  misc_feature 1..20: note = seed sequence RRA
  source 1..20: mol_type = other DNA
tattgtatgcctgggatgac